(19) United States
(12) Patent Application Publication
(10) Pub. No.: US 2024/0425572 A1
(43) Pub. Date: Dec. 26, 2024

(54) ANTIBODIES AND METHODS OF USING THEREOF

(71) Applicant: REGENXBIO Inc., Rockville, MD (US)

(72) Inventors: Michele Fiscella, Rockville, MD (US); Lin Yang, Rockville, MD (US); Zhuchun WU, North Potomac, MD (US); Hiren Patel, Rockville, MD (US); Hua Wang, Rockville, MD (US)

(21) Appl. No.: 18/701,201

(22) PCT Filed: Oct. 15, 2022
(86) PCT No.: PCT/US2022/078180

§ 371 Date: Apr. 12, 2024

Related U.S. Application Data
(60) Provisional application No. 63/256,511, filed on Oct. 15, 2021.

(51) Int. Cl. C07K 16/18; G01N 33/68

(52) U.S. Cl. CPC C07K 16/18; G01N 33/6848; G01N 33/6887; C07K 2317/92; G01N 2458/15; G01N 2800/2885

(57) ABSTRACT

Provided herein are antibodies that specifically bind to a peptide antigen. Also provided are methods of detecting, isolating or quantifying the peptide antigen. In some embodiments, the peptide antigen is released from dystrophin or microdystrophin by protease digestion. Also provided are methods for detecting or quantifying microdystrophin or dystrophin in a sample using the antibodies. In some embodiments, a method described herein is used to monitor the expression of microdystrophin in a subject that has been administered a nucleic acid based therapy.

### CROSS-REFERENCE TO RELATED APPLICATIONS

This application claims the benefit of U.S. application No. 63/256,511, filed Oct. 15, 2021, which is incorporated herein by reference in its entirety.

### TECHNICAL FIELD

The present disclosure relates to antibodies that specifically bind to a peptide antigen and can be used to detect, isolate or quantify the peptide antigen.

### REFERENCE TO SEQUENCE LISTING SUBMITTED ELECTRONICALLY

The content of the electronically submitted sequence listing (Name: 6728_1401_Sequence_Listing.xml; Size: 29,401 bytes; and Date of Creation: Oct. 13, 2022) filed with the application is incorporated herein by reference in its entirety.

### BACKGROUND

A group of neuromuscular diseases called dystrophinopathies are caused by mutations in the DMD gene. Each dystrophinopathy has a distinct phenotype, with all patients suffering from muscle weakness and ultimately cardiomyopathy with ranging severity. Duchenne muscular dystrophy (DMD) is caused by frameshift mutations in the dystrophin gene abolishing the expression of the dystrophin protein. Due to the lack of the dystrophin protein, skeletal muscle, and ultimately heart and respiratory muscles (e.g., intercostal muscles and diaphragm), degenerate causing premature death. Progressive weakness and muscle atrophy begin in childhood. Affected individuals experience breathing difficulties, respiratory infections, and swallowing problems. Almost all DMD patients will develop cardiomyopathy. Pneumonia compounded by cardiac involvement is the most frequent cause of death, which frequently occurs before the third decade. Becker muscular dystrophy (BMD) has less severe symptoms than DMD, but still leads to premature death.

Dystrophin is a cytoplasmic protein encoded by the DMD gene, which is the largest known human gene. Full-length dystrophin is a large (427 kDa) protein comprising a number of subdomains that contribute to its function. In DMD, mutations often lead to a frame shift resulting in a premature stop codon and a truncated, non-functional or unstable protein. In BMD, patients express a truncated, partially functional dystrophin.

Adeno-associated virus (AAV) mediated gene therapy is being developed for the treatment of DMD, BMD and less severe dystrophinopathies. Due to limits on payload size of AAV vectors, attention has focused on creating micro- or mini-dystrophins, smaller versions of dystrophin that eliminate non-essential subdomains while maintaining at least some function of the full-length protein. Int'l. Appl. Pub. No. WO 2021108755.

There is a need for methods of detecting and quantifying transgene mediated micro- and minidystrophin expression in subjects receiving gene therapy.

### BRIEF SUMMARY

In one aspect, provided herein is an isolated antibody or antigen binding fragment thereof capable of binding to a polypeptide comprising the amino acid sequence of SEQ ID NO: 1. In some embodiments, the antibody comprises the 6 complementarity-determining regions (CDRs) of 130D2-1, 133E10-1 or 75A2-1. In some embodiments, the antibody comprises the VH and VL domains of 130D2-1, 133E10-1 or 75A2-1. In some embodiments, the antibody is 130D2-1, 133E10-1 or 75A2-1. In some embodiments, the antibody is 130D2-1. In some embodiments, the CDRs are according to Kabat. In some embodiment, the antibody comprises an antigen-binding antibody fragment.

In one aspect, provided herein is an isolated antibody or antigen binding fragment thereof capable of binding to a polypeptide comprising the amino acid sequence of SEQ ID NO: 3. In some embodiments, the antibody comprises the 6 complementarity-determining regions (CDRs) of 112E4-1, 115G6-1, 119H2-1, 121F10-1 or 133D7-1. In some embodiments, the antibody comprises the VH and VL domains of 112E4-1, 115G6-1, 119H2-1, 121F10-1 or 133D7-1. In some embodiments, the antibody is 112E4-1, 115G6-1, 119H2-1, 121F10-1 or 133D7-1. In some embodiments, the antibody is 133D7-1. In some embodiments, the CDRs are according to Kabat. In some embodiment, the antibody comprises an antigen-binding antibody fragment.

In one aspect, provided herein is a composition comprising an antibody described herein. In some embodiments, the antibody is 130D2-1. In some embodiments, the antibody is 133D7-1.

In one aspect, provided herein is an affinity resin comprising an antibody described herein and a solid support. In some embodiments, the antibody is 130D2-1. In some embodiments, the antibody is 133D7-1.

In one aspect, provided herein is an isolated polynucleotide encoding an antibody described herein. In some embodiments, the antibody is 130D2-1. In some embodiments, the antibody is 133D7-1.

In one aspect, provided herein is a method of producing an antibody disclosed herein comprising incubating a host cell comprising a polynucleotide encoding the antibody under suitable conditions to produce the antibody. In some embodiments, the antibody is 130D2-1. In some embodiments, the antibody is 133D7-1.

In one aspect, provided herein is a method of detecting, isolating or quantifying a peptide having the sequence of SEQ ID NO: 1 or 3 in a sample comprising contacting the sample comprising the peptide with an antibody described herein under conditions that permit binding of the peptide to the antibody. In some embodiments, the method further comprises recovering the peptide. In some embodiments, the method further comprises determining the amount of peptide recovered. In some embodiments, the amount of peptide is determined by LC/MS or LC-MS/MS. In some embodiments, the amino acid sequence of the peptide comprises SEQ ID NO: 1 and the antibody is 130D2-1. In some embodiments, the amino acid sequence of the peptide comprises SEQ ID NO: 3 and the antibody is 133D7-1.

In one aspect, provided herein is a method of detecting or quantifying the level of a recombinant polypeptide in a sample comprising contacting the sample with an antibody described herein under conditions that permit binding of a peptide having the sequence of SEQ ID NO: 1 or 3 to the antibody, wherein the sample comprises a protease digested protein isolate, and wherein the amino acid sequence of the recombinant polypeptide comprises SEQ ID NO: 28 and/or 29. In some embodiments, the amino acid sequence of the recombinant polypeptide comprises SEQ ID NO: 1 and the antibody is 130D2-1. In some embodiments, the amino acid sequence of the recombinant polypeptide comprises SEQ ID NO: 3 and the antibody is 133D7-1.

In one aspect, provided herein is a method of detecting or quantifying the level of dystrophin and/or microdystrophin in a sample comprising contacting the sample with an antibody described herein under conditions that permit binding of a peptide having the sequence of SEQ ID NO: 1 or 3 to the antibody, wherein the sample comprises a protease digested protein isolate, and wherein the amino acid sequence of the microdystrophin comprises SEQ ID NO: 28 and/or 29. In some embodiments the dystrophin is human, primate or murine dystrophin. In some embodiments, the microdystrophin comprises the amino acid sequence of SEQ ID NO: 27. In some embodiments, the sample comprises is a protease digested protein isolate from a subject that has been administered a recombinant adeno-associated virus comprising a polynucleotide encoding the microdystrophin. In some embodiments, the protein detected or quantified is microdystrophin, and the antibody is 130D2-1. In some embodiments, the protein detected or quantified is microdystrophin and dystrophin, and the antibody is 133D7-1.

In some embodiments, the disclosure provides:
[1.] An isolated antibody or antigen binding fragment thereof capable of binding to a polypeptide comprising the amino acid sequence of SEQ ID NO: 1;
[2.] the antibody or antigen binding fragment thereof of [1], wherein the amino acid sequence of the polypeptide consist of SEQ ID NO: 1;
[3.] the antibody or antigen binding fragment thereof of [1] or [2] that does not bind to a peptide consisting of the amino acid of SEQ ID NO: 2;
[4.] the antibody or antigen binding fragment thereof of [1] to [3], wherein the antibody is a polyclonal antibody;
[5.] the antibody or antigen binding fragment thereof of [1] to [3], wherein the antibody is a monoclonal antibody;
[6.] An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
a) the VH CDR1, CDR2 and CDR3 and the VL CDR1, CDR2 and CDR3 of the 130D2-1 antibody, respectively, independently comprising 0, 1, 2, 3, 4 or 5 substitutions;
b) the VH CDR1, CDR2 and CDR3 and the VL CDR1, CDR2 and CDR3 of the 133E10-1 antibody, respectively, independently comprising 0, 1, 2, 3, 4 or 5 substitutions; or
c) the VH CDR1, CDR2 and CDR3 and the VL CDR1, CDR2 and CDR3 of the 75A2-1 antibody, respectively, independently comprising 0, 1, 2, 3, 4 or 5 substitutions;
[7.] An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
a) the VH CDR1, CDR2 and CDR3 and the VL CDR1, CDR2 and CDR3 of the 130D2-1 antibody, respectively;
b) the VH CDR1, CDR2 and CDR3 and the VL CDR1, CDR2 and CDR3 of the 133E10-1 antibody, respectively; or
c) the VH CDR1, CDR2 and CDR3 and the VL CDR1, CDR2 and CDR3 of the 75A2-1 antibody, respectively;
[8.] the isolated antibody or antigen binding fragment thereof according to [6] or [7], wherein the VH CDR1, CDR2 and CDR3 and the VL CDR1, CDR2 and CDR3 of 130D2-1, 133E10-1 and 75A2-1 are according to Kabat;
[9.] An isolated antibody or antigen binding fragment thereof comprising a variable heavy chain domain (VH) and a variable light chain domain (VL), wherein the VH and VL comprises
a) an amino acid sequence having at least 70%, at least 80%, at least 90%, at least 95%, at least 97% or 100% identity with the VH and VL of the 130D2-1 antibody, respectively;
b) an amino acid sequence having at least 70%, at least 80%, at least 90%, at least 95%, at least 97% or 100% identity with the VH and VL of the 133E10-1 antibody, respectively; or
c) an amino acid sequence having at least 70%, at least 80%, at least 90%, at least 95%, at least 97% or 100% identity with the VH and VL of the 75A2-1 antibody, respectively;
[10.] An isolated antibody or antigen binding fragment thereof comprising a variable heavy chain domain (VH) and a variable light chain domain (VL), wherein the VH and VL comprises
a) the VH and VL of the 130D2-1 antibody, respectively;
b) the VH and VL of the 133E10-1 antibody, respectively; or
c) the VH and VL of the 75A2-1 antibody, respectively;
[11.] An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the
a) the VH CDR1 comprises an amino acid sequence of SEQ ID NO: 8 comprising 0, 1, 2, 3, 4 or 5 substitutions;
b) the VH CDR2 comprises an amino acid sequence of SEQ ID NO: 9 comprising 0, 1, 2, 3, 4 or 5 substitutions;
c) the VH CDR3 comprises an amino acid sequence of SEQ ID NO: 10 comprising 0, 1, 2, 3, 4 or 5 substitutions;
d) the VL CDR1 comprises an amino acid sequence of SEQ ID NO: 11 comprising 0, 1, 2, 3, 4 or 5 substitutions;
e) the VL CDR2 comprises an amino acid sequence of SEQ ID NO: 12 comprising 0, 1, 2, 3, 4 or 5 substitutions; and
f) the VL CDR3 comprises an amino acid sequence of SEQ ID NO: 13 comprising 0, 1, 2, 3, 4 or 5 substitutions;
[12.] An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the
a) the VH CDR1 comprises the amino acid sequence of SEQ ID NO: 8;
b) the VH CDR2 comprises the amino acid sequence of SEQ ID NO: 9;
c) the VH CDR3 comprises the amino acid sequence of SEQ ID NO: 10;
d) the VL CDR1 comprises the amino acid sequence of SEQ ID NO: 11;
e) the VL CDR2 comprises the amino acid sequence of SEQ ID NO: 12; and
f) the VL CDR3 comprises the amino acid sequence of SEQ ID NO: 13;
[13.] An isolated antibody or antigen binding fragment thereof comprising a variable heavy chain domain (VH) and a variable light chain domain (VL), wherein
a) the VH comprises an amino acid sequence having at least 70%, at least 80%, at least 90%, at least 95%, at least 97% or 100% identity with SEQ ID NO: 4; and
b) the VL comprises an amino acid sequence having at least 70%, at least 80%, at least 90%, at least 95%, at least 97% or 100% identity with SEQ ID NO: 5;
[14.] An isolated antibody or antigen binding fragment thereof comprising a variable heavy chain domain (VH) and a variable light chain domain (VL), wherein
a) the VH comprises the amino acid sequence of SEQ ID NO: 4; and
b) the VL comprises the amino acid sequence of SEQ ID NO: 5;
[15.] the antibody or antigen binding fragment thereof of [6] to capable of binding to a polypeptide comprising the amino acid sequence of SEQ ID NO: 1;
[16.] the antibody or antigen binding fragment thereof of [15], wherein the amino acid sequence of the polypeptide consist of SEQ ID NO: 1;
[17.] the antibody or antigen binding fragment thereof of [6] to that does not bind to a peptide consisting of the amino acid of SEQ ID NO: 2;
[18.] An isolated antibody or antigen binding fragment thereof capable of binding to a polypeptide comprising the amino acid sequence of SEQ ID NO: 3;
[19.] the antibody or antigen binding fragment thereof of [18], wherein the amino acid sequence of the polypeptide consist of SEQ ID NO: 3;
[20.] the antibody or antigen binding fragment thereof of or [19], wherein the antibody is a polyclonal antibody;
[21.] the antibody or antigen binding fragment thereof of or [19], wherein the antibody is a monoclonal antibody;
[22.] An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
a) the VH CDR1, CDR2 and CDR3 and the VL CDR1, CDR2 and CDR3 of the 112E4-1 antibody, respectively, independently comprising 0, 1, 2, 3, 4 or 5 substitutions;
b) the VH CDR1, CDR2 and CDR3 and the VL CDR1, CDR2 and CDR3 of the 115G6-1 antibody, respectively, independently comprising 0, 1, 2, 3, 4 or 5 substitutions;
c) the VH CDR1, CDR2 and CDR3 and the VL CDR1, CDR2 and CDR3 of the 119H2-1 antibody, respectively, independently comprising 0, 1, 2, 3, 4 or 5 substitutions;
d) the VH CDR1, CDR2 and CDR3 and the VL CDR1, CDR2 and CDR3 of the 121F10-1 antibody, respectively, independently comprising 0, 1, 2, 3, 4 or 5 substitutions; or
e) the VH CDR1, CDR2 and CDR3 and the VL CDR1, CDR2 and CDR3 of the 133D7-1 antibody, respectively, independently comprising 0, 1, 2, 3, 4 or 5 substitutions;
[23.] An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
a) the VH CDR1, CDR2 and CDR3 and the VL CDR1, CDR2 and CDR3 of the 112E4-1 antibody, respectively;
b) the VH CDR1, CDR2 and CDR3 and the VL CDR1, CDR2 and CDR3 of the 115G6-1 antibody, respectively;
c) the VH CDR1, CDR2 and CDR3 and the VL CDR1, CDR2 and CDR3 of the 119H2-1 antibody, respectively;
d) the VH CDR1, CDR2 and CDR3 and the VL CDR1, CDR2 and CDR3 of the 121F10-1 antibody, respectively; or
e) the VH CDR1, CDR2 and CDR3 and the VL CDR1, CDR2 and CDR3 of the 133D7-1 antibody, respectively;
[24.] the isolated antibody or antigen binding fragment thereof according to or [23], wherein the VH CDR1, CDR2 and CDR3 and the VL CDR1, CDR2 and CDR3 of 112E4-1, 115G6-1, 119H2-1, 121F10-1 and 133D7-1 are according to Kabat;
[25.] An isolated antibody or antigen binding fragment thereof comprising a variable heavy chain domain (VH) and a variable light chain domain (VL), wherein the VH and VL comprises
a) an amino acid sequence having at least 70%, at least 80%, at least 90%, at least 95%, at least 97% or 100% identity with the VH and VL of the 112E4-1 antibody, respectively;
b) an amino acid sequence having at least 70%, at least 80%, at least 90%, at least 95%, at least 97% or 100% identity with the VH and VL of the 115G6-1 antibody, respectively;
c) an amino acid sequence having at least 70%, at least 80%, at least 90%, at least 95%, at least 97% or 100% identity with the VH and VL of the 119H2-1 antibody, respectively;
d) an amino acid sequence having at least 70%, at least 80%, at least 90%, at least 95%, at least 97% or 100% identity with the VH and VL of the 121F10-1 antibody, respectively; or
e) an amino acid sequence having at least 70%, at least 80%, at least 90%, at least 95%, at least 97% or 100% identity with the VH and VL of the 133D7-1 antibody, respectively;
[26.] An isolated antibody or antigen binding fragment thereof comprising a variable heavy chain domain (VH) and a variable light chain domain (VL), wherein the VH and VL comprises
a) the VH and VL of the 112E4-1 antibody, respectively;
b) the VH and VL of the 115G6-1 antibody, respectively;
c) the VH and VL of the 119H2-1 antibody, respectively;
d) the VH and VL of the 121F10-1 antibody, respectively; or
e) the VH and VL of the 133D7-1 antibody, respectively;
[27.] An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the
a) the VH CDR1 comprises an amino acid sequence of SEQ ID NO: 18 comprising 0, 1, 2, 3, 4 or 5 substitutions;
b) the VH CDR2 comprises an amino acid sequence of SEQ ID NO: 19 comprising 0, 1, 2, 3, 4 or 5 substitutions;
c) the VH CDR3 comprises an amino acid sequence of SEQ ID NO: 20 comprising 0, 1, 2, 3, 4 or 5 substitutions;
d) the VL CDR1 comprises an amino acid sequence of SEQ ID NO: 21 comprising 0, 1, 2, 3, 4 or 5 substitutions;
e) the VL CDR2 comprises an amino acid sequence of SEQ ID NO: 22 comprising 0, 1, 2, 3, 4 or 5 substitutions; and
f) the VL CDR3 comprises an amino acid sequence of SEQ ID NO: 23 comprising 0, 1, 2, 3, 4 or 5 substitutions;
[28.] An isolated antibody or antigen binding fragment thereof comprising a variable heavy chain domain (VH) and a variable light chain domain (VL), wherein the VH comprises VH complementarity determining regions (CDRs) 1, 2 and 3 and the VL comprises VL CDRs 1, 2 and 3, wherein the
a) the VH CDR1 comprises the amino acid sequence of SEQ ID NO: 18;
b) the VH CDR2 comprises the amino acid sequence of SEQ ID NO: 19;
c) the VH CDR3 comprises the amino acid sequence of SEQ ID NO: 20;
d) the VL CDR1 comprises the amino acid sequence of SEQ ID NO: 21;
e) the VL CDR2 comprises the amino acid sequence of SEQ ID NO: 22; and
f) the VL CDR3 comprises the amino acid sequence of SEQ ID NO: 23;
[29.] An isolated antibody or antigen binding fragment thereof comprising a variable heavy chain domain (VH) and a variable light chain domain (VL), wherein
a) the VH comprises an amino acid sequence having at least 70%, at least 80%, at least 90%, at least 95%, at least 97% or 100% identity with SEQ ID NO: 14; and
b) the VL comprises an amino acid sequence having at least 70%, at least 80%, at least 90%, at least 95%, at least 97% or 100% identity with SEQ ID NO: 15;
[30.] An isolated antibody or antigen binding fragment thereof comprising a variable heavy chain domain (VH) and a variable light chain domain (VL), wherein
a) the VH comprises the amino acid sequence of SEQ ID NO: 14; and
b) the VL comprises the amino acid sequence of SEQ ID NO: 15;
[31.] the antibody or antigen binding fragment thereof of to capable of binding to a polypeptide comprising the amino acid sequence of SEQ ID NO: 3;
[32.] the antibody or antigen binding fragment thereof of [31], wherein the amino acid sequence of the polypeptide consist of SEQ ID NO: 3;
[33.] the antibody or antigen binding fragment thereof of [1] to [32], wherein the antibody fragment comprises a single-chain Fv (scFv), F (ab) fragment, F(ab′) 2 fragment, or an isolated VH domain;
[34.] A composition comprising the antibody or antigen binding fragment thereof of [1] to [33];
[35.] the composition of that is a pharmaceutical composition further comprising a pharmaceutically acceptable excipient;
[36.] An affinity resin comprising the antibody or antigen binding fragment thereof of [1] to and a solid support;
[37.] the affinity resin of [36], wherein the solid support comprises a bead, gelatin, or agarose;
[38.] the affinity resin of or [37], wherein the antibody or antigen binding fragment thereof is attached to the solid support by covalent bonding;
[39.] the affinity resin of or [37], wherein the antibody or antigen binding fragment thereof is attached to the solid support by non-covalent association;
[40.] An isolated polynucleotide encoding the antibody or antigen binding fragment thereof of [1] to [33];
[41.] A vector comprising the polynucleotide of [40];
[42.] A host cell comprising the polynucleotide of [40 or the vector of [41];
[43.] the host cell of which is a CHO cell or a HEK293 cell;
[44.] A method of producing the antibody or antigen binding fragment thereof of [1] to comprising incubating the host cell of or under suitable conditions to produce the antibody or antigen binding fragment thereof;
[45.] A method of isolating a peptide from a sample comprising
a) contacting the sample comprising the peptide with a composition comprising the antibody or antigen binding fragment thereof of [1] to [17], and optionally with a composition comprising the antibody or antigen binding fragment thereof of any one for claims to [33],] under conditions that permit binding of the peptide to the antibody or antigen binding fragment thereof;
b) removing a portion of the sample that is not bound to the antibody or antigen binding fragment thereof; and
c) dissociating the peptide from the antibody or antigen binding fragment thereof, wherein the amino acid sequence of the peptide comprises SEQ ID NO: 1 or 3;
[46.] the method of [45], wherein the amino acid sequence of the peptide consist of SEQ ID NO: 1 or 3;
[47.] the method of or [46], wherein the composition comprising the antibody or antigen binding fragment thereof is an affinity resin comprising a solid support selected from the group consisting of a bead, gelatin, or agarose;
[48.] the method of [47], wherein the antibody or antigen binding fragment thereof is attached to the solid support by covalent bonding;
[49.] the method of [47], wherein the antibody or antigen binding fragment thereof is attached to the solid support by non-covalent association;
[50.] the method of to [49], wherein the sample comprises a protease digested protein isolate obtained from a subject;
[51.] the method of [50], wherein the sample comprises a protease digested protein isolate obtained from a skeletal muscle tissue of the subject;
[52.] the method of or [51], wherein the protease comprises trypsin;
[53.] the method of to [52], wherein the subject is a human, primate, canine or murine subject;
[54.] the method of to [53], wherein the subject has been administered a recombinant polypeptide comprising the amino acid sequence of SEQ ID NO: 28 and/or 29;
[55.] the method of to [53], wherein the subject has been administered a recombinant polynucleotide encoding a recombinant polypeptide comprising the amino acid sequence of SEQ ID NO: 28 and/or 29;
[56.] the method of to [53], wherein the subject has been administered a recombinant virus comprising a polynucleotide encoding a recombinant polypeptide comprising the amino acid sequence of SEQ ID NO: 28 and/or 29;
[57.] the method of [56], wherein the recombinant virus is a recombinant adeno-associated virus;
[58.] the method of to [57], further comprising
d) recovering the peptide; and
e) determining the amount of peptide recovered in step d);
[59.] the method of [58], wherein the amount of peptide is determined by LC/MS or LC-MS/MS;
[60.] the method of to [59], wherein the sample further comprises a stable isotope labeled peptide standard comprising the amino acid sequence of SEQ ID NO: 1 or 3;
[61.] the method of to [60], wherein the recombinant polypeptide is microdystrophin;
[62.] the method of [61], wherein the microdystrophin comprises the amino acid sequence of SEQ ID NO: 27;
[63.] A method of quantifying the level of a recombinant polypeptide in a subject comprising:
a) providing a sample comprising a protease digested protein isolate obtained from the subject, wherein the sample comprises one or more peptides having the amino acid sequence of SEQ ID NO: 1 or 3;
b) contacting the sample with a composition comprising the antibody or antigen binding fragment thereof of [1] to [17], and optionally with a composition comprising the antibody or antigen binding fragment thereof of any one for claims to [33], under conditions that permit binding of the antibody or antigen binding fragment thereof to the peptide;
c) recovering the peptide bound to the antibody or antigen binding fragment thereof; and
d) determining the amount of peptide recovered in step d), wherein the amino acid sequence of the recombinant polypeptide comprises SEQ ID NO: 28 and/or 29;
[64.] the method of [63], wherein the amount of peptide is determined by LC/MS or LC-MS/MS;
[65.] the method of or [64], wherein the sample comprises a protease digested protein isolate obtained from a skeletal muscle tissue of the subject;
[66.] the method of to [65], wherein the subject is a human, primate, canine or murine subject;
[67.] the method of to [66], wherein the subject has been administered the recombinant polypeptide comprising the amino acid sequence of SEQ ID NO: 28 and/or 29;
[68.] the method of to [66], wherein the subject has been administered a recombinant polynucleotide encoding a recombinant polypeptide comprising the amino acid sequence of SEQ ID NO: 28 and/or 29;
[69.] the method of to [66], wherein the subject has been administered a recombinant virus comprising a polynucleotide encoding a recombinant polypeptide comprising the amino acid sequence of SEQ ID NO: 28 and/or 29;
[70.] the method of [69], wherein the recombinant virus is a recombinant adeno-associated virus;
[71.] the method of to [70], wherein the recombinant polypeptide is microdystrophin;
[72.] the method of [71], wherein the microdystrophin comprises the amino acid sequence of SEQ ID NO: 27;
[73.] the method of any one of to [72], wherein the sample further comprises a labeled peptide or peptides that are capable of binding to the antibody or antigen binding fragment thereof;
[74.] the method of [73], wherein the labeled peptide or peptides are a stable isotope labeled peptide or peptides;
[75.] A method of quantifying the level of dystrophin and/or microdystrophin expression in a subject comprising:
a) providing a sample comprising a protease digested protein isolate obtained from the subject, wherein the sample comprises one or more peptides having the amino acid sequence of SEQ ID NO: 1 or 3;
b) contacting the sample with a composition comprising the antibody or antigen binding fragment thereof of [1] to [17], and optionally with a composition comprising the antibody or antigen binding fragment thereof of any one for claims to [33], under conditions that permit binding of the antibody or antigen binding fragment thereof to the peptide;
c) recovering the peptide bound to the antibody or antigen binding fragment thereof; and
d) determining the amount of peptide recovered in step d), wherein the amino acid sequence of the microdystrophin comprises SEQ ID NO: 28 and/or 29;
[76.] the method of [73], wherein the amount of peptide is determined by LC/MS or LC-MS/MS;
[77.] the method of or [74], which provides an absolute quantification of the level of dystrophin and/or microdystrophin expression;
[78.] the method of or [74], which provides a relative quantification of the level of dystrophin and/or microdystrophin expression;
[79.] the method of to [76], wherein the sample comprises a protease digested protein isolate obtained from a skeletal muscle tissue of the subject;
[80.] the method of to [77], wherein the protease is trypsin;
[81.] the method of any one of to [80], wherein the sample further comprises a labeled peptide or peptides that are capable of binding to the antibody or antigen binding fragment thereof;
[82.] the method of [81], wherein the labeled peptide or peptides are a stable isotope labeled peptide or peptides;
[83.] the method of or [82], wherein the labeled peptide or peptides comprise the amino acid sequence of SEQ ID NO: 1 or 3;
[84.] the method of to [83], wherein the subject is a human, primate, canine or murine subject;
[85.] the method of [84], wherein the subject is a human;
[86.] the method of [84], wherein the subject is a primate;
[87.] the method of [84], wherein the subject is a murine;
[88.] the method of to [84], wherein the subject suffers from Duchenne muscular dystrophy;
[89.] the method of to [84], wherein the subject is a non-human mammal that has been genetically modified to comprise one or more mutations in the dystrophin gene;
[90.] the method of to [89], wherein the subject has been administered a recombinant polynucleotide encoding a microdystrophin comprising the amino acid sequence of SEQ ID NO: 28 and 29;
[91.] the method of [90], wherein the microdystrophin comprises the amino acid sequence of SEQ ID NO: 27;
[92.] the method of or [91], wherein the recombinant polynucleotide is DNA;
[93.] the method of or [91], wherein the recombinant polynucleotide is RNA;
[94.] the method of [93], wherein the RNA is mRNA comprising a modified ribonucleotide;
[95.] the method of to [89], wherein the subject has been administered a recombinant virus comprising a polynucleotide encoding a microdystrophin comprising the amino acid sequence of SEQ ID NO: 28 and 29;
[96.] the method of [69], wherein the microdystrophin comprises the amino acid sequence of SEQ ID NO: 27;
[97.] the method of or [96], wherein the recombinant virus is a recombinant adeno-associated virus.

Still other features and advantages of the compositions and methods described herein will become more apparent from the following detailed description when read in conjunction with the accompanying drawings.

### BRIEF DESCRIPTION OF THE DRAWINGS

FIG. 1. Candidate tryptic peptides for LC-MS quantification of microdystrophin A.

FIG. 2. Binding of rabbit monoclonal antibodies and rabbit polyclonal antibodies to LEM peptide.

FIG. 3. Binding of rabbit monoclonal antibodies and rabbit polyclonal antibodies to LLQ peptide.

FIG. 4. Rabbit anti-LEM peptide monoclonal antibody screening by LCMS.

FIG. 5. Rabbit anti-LLQ peptide monoclonal antibody screening by LCMS.

FIG. 6. LCMS Assay for simultaneous quantification of microdystrophin A and dystrophin.

FIG. 7. LBA/LC-MS/MS Assay workflow.

FIG. 8. Selectivity of LEM peptide detection assay.

FIG. 9. Selectivity of LLQ peptide detection assay.

FIG. 10. Absolute quantification of microdystrophin transgene product by LEM peptide detection assay.

FIG. 11. Relative quantification of full-length Dystrophin in various species by LLQ peptide detection assay.

### DETAILED DESCRIPTION

Provided herein are antibodies that specifically bind to a peptide having the sequence of SEQ ID NO: 1 or 3. The antibodies are useful for the detection, isolation and quantitation of a peptide having the sequence of SEQ ID NO: 1 or 3. The antibodies can also be used for detecting or quantifying a protein, for example, dystrophin or microdystrophin, whose digestion with a protease, e.g., trypsin, releases a peptide having the sequence of SEQ ID NO: 1 or 3.

In some embodiments, provided herein are methods for detecting and quantifying dystrophin and/or microdystrophin expression in a subject suffering from muscular dystrophy that has been administered a recombinant polynucleotide encoding a microdystrophin. In some embodiments, the subject has been administered a recombinant adeno-associated virus comprising the recombinant polynucleotide encoding the microdystrophin. In some embodiments, the method uses Liquid Chromatography-Mass Spectrometry (LC-MS) or the Liquid Chromatography-Tandem Mass Spectrometry (LC-MS/MS) to detect and quantify peptides comprising the amino acid sequence of SEQ ID NO: 1 or 3. In some embodiments, the LC-MS detection assay disclosed herein provides highly accurate absolute quantification of microdystrophin levels in muscle samples of non-human primate subjects dosed with recombinant AAV particles comprising a microdystrophin transgene. In some embodiments, the LC-MS detection assay disclosed herein also provides highly accurate relative quantification of total dystrophin levels in muscle samples from various species, e.g., humans and primates.

### Definitions

Unless defined otherwise, all technical and scientific terms used herein have the same meaning as commonly understood by one of ordinary skill in the art to which this disclosure is related. To facilitate an understanding of the disclosed methods, a number of terms and phrases are defined below.

The term “antibody” refers to an immunoglobulin molecule (or a group of immunoglobulin molecules) that recognizes and specifically binds to a target, such as a protein, polypeptide, peptide, carbohydrate, polynucleotide, lipid, or combinations of the foregoing through at least one antigen recognition site within the variable region of the immunoglobulin molecule. The terms “antibody” and “antibodies” are terms of art and can be used interchangeably herein and refer to a molecule with an antigen-binding site that specifically binds an antigen.

Antibodies can include, for example, monoclonal antibodies, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), affybodies, Fab fragments, F(ab′) 2 fragments, disulfide-linked Fvs (sdFv), anti-idiotypic (anti-Id) antibodies (including, e.g., anti-anti-Id antibodies), bispecific antibodies, and multi-specific antibodies. In certain embodiments, antibodies described herein refer to polyclonal antibody populations. Antibodies can be of any type (e.g., IgG, IgE, IgM, IgD, IgA, or IgY), any class (e.g., IgG1, IgG2, IgG3, IgG4, IgA1, or IgA2), or any subclasses (isotypes) thereof (e.g. IgG1, IgG2, IgG3, IgG4, IgA1 and IgA2), of immunoglobulin molecule, based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated or fused to other molecules such as toxins, radioisotopes, other polypeptides etc.

The terms “antigen-binding domain,” “antigen-binding region,” “antigen-binding site,” and similar terms refer to the portion of antibody molecules, which comprises the amino acid residues that confer on the antibody molecule its specificity for the antigen (e.g., peptide having the sequence of SEQ ID NO: 1 or 3). The antigen-binding region can be derived from any animal species, such as mouse and humans.

The terms “variable region” or “variable domain” are used interchangeably and are common in the art. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen (e.g., peptide having the sequence of SEQ ID NO: 1 or 3). In certain embodiments, the variable region comprises 3 CDRs (CDR1, CDR2, and CDR3) and 4 framework regions (FR1, FR2, FR3, and FR4) in the order of FR1-CDR1-FR2-CDR2-FR3-CDR3-FR4 from the N terminus to the C terminus. In certain embodiments, the variable region is a human variable region. In certain embodiments, the variable region comprises human CDRs and human framework regions (FRs). In certain embodiments, the variable region comprises CDRs and framework regions (FRs) wherein one or more of the CDRs were modified by a substitution, deletion, or insertion relative to the CDRs of a parental antibody. In certain embodiments, the variable region comprises CDRs and framework regions (FRs) wherein one or more of the FRs were modified by a substitution, deletion, or insertion relative to the FRs of a parental antibody. In certain embodiments, the variable region comprises CDRs and framework regions (FRs) wherein one or more of the CDRs and one or more of the FRs were modified by a substitution, deletion, or insertion relative to the CDRs and FRs of a parental antibody. In certain embodiments, the parental antibody is PGZL1. In certain embodiments, the variable region comprises human CDRs and primate (e.g., non-human primate) framework regions (FRs).

A skilled artisan understands that there are several methods for determining CDRs. One approach is based on cross-species sequence variability (i.e., Kabat E A, et al., Sequences of Proteins of Immunological Interest, (5th ed., 1991, National Institutes of Health, Bethesda Md.) (“Kabat”). Another approach is based on crystallographic studies of antigen-antibody complexes (Al-lazikani B., et al, J. Mol. Biol. 273:927-948 (1997)) (“Chothia”). In addition, combinations of these two approaches are sometimes used in the art to determine CDRs. In some embodiments, the CDR sequences are identified according to Kabat. In some embodiments, the CDR sequences are identified according to Chothia. It is understood that the identification of CDRs in a variable region also identifies the FRs as the sequences flanking the CDRs.

The Kabat numbering system is generally used when referring to a residue in the variable domain (approximately residues 1-107 of the light chain and residues 1-113 of the heavy chain) (e.g., Kabat E A, et al., Sequences of Immunological Interest. (5th Ed., 1991, National Institutes of Health, Bethesda, Md.) (“Kabat”).

The amino acid position numbering as in Kabat, refers to the numbering system used for heavy chain variable domains or light chain variable domains of the compilation of antibodies in Kabat E A, et al. (Sequences of Immunological Interest. (5th Ed., 1991, National Institutes of Health, Bethesda, Md.), “Kabat”). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software, available, for example, at bioinf.org.uk/abs/software. In some embodiments, the CDR sequences are identified according to Kabat. In some embodiments, the CDR sequences are identified according to Chothia. In some embodiments, the CDR sequences are identified according to AbM. In some embodiments, the VH CDR3 sequence is identified according to Kabat. In some embodiments, the VH CDR3 sequence is identified according to Chothia. In some embodiments, the VH CDR3 sequence is identified according to AbM.

| Loop | Kabat | AbM | Chothis |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |
| | | (Kabat Numbering) | |
| H1 | H31-H35 | H26-H35 | H26-H32 |
| | | (Chothia Numbering) | |
| H2 | H50-H65 | H50-H58 | H52-H56 |
| H3 | H95-H102 | H95-H102 | H95-H102 |

The terms “VL” and “VL domain” are used interchangeably to refer to the light chain variable region of an antibody.

The terms “VH” and “VH domain” are used interchangeably to refer to the heavy chain variable region of an antibody.

The term “antibody fragment” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′) 2, and Fv fragments, linear antibodies, and single chain antibodies.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′) 2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

A polypeptide, antibody, polynucleotide, vector, cell, or composition, which is “isolated” is a polypeptide, antibody, polynucleotide, vector, cell, or composition, which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition, which is isolated is substantially pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides described herein are based upon antibodies, in certain embodiments, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin S., et al, Proc. Natl. Acad. Sci., 87:2264-2268 (1990), as modified in Karlin S., et al., Proc. Natl. Acad. Sci., 90:5873-5877 (1993), and incorporated into the NBLAST and XBLAST programs (Altschul S F, et al., Nucleic Acids Res., 25:3389-3402 (1991)). In certain embodiments, Gapped BLAST can be used as described in Altschul S F, et al., Nucleic Acids Res. 25:3389-3402 (1997). BLAST-2, WU-BLAST-2 (Altschul S F, et al., Methods in Enzymology, 266:460-480 (1996)), ALIGN, ALIGN-2 (Genentech, South San Francisco, California) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48): 444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, WI 53711). Bestfit uses the local homology algorithm of Smith and Waterman (Advances in Applied Mathematics 2:482 489 (1981)) to find the best segment of homology between two sequences. When using Bestfit or any other sequence alignment program to determine whether a particular sequence is, for instance, 95% identical to a reference sequence described herein, the parameters are set such that the percentage of identity is calculated over the full length of the reference nucleotide sequence and that gaps in identity of up to 5% of the total number of nucleotides in the reference sequence are allowed.

In some embodiments, two nucleic acids or polypeptides described herein are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value there between, and can be over a longer region than 60-80 residues, for example, at least about 90-100 residues, and in some embodiments, the sequences are substantially identical over the full length of the sequences being compared, such as the coding region of a nucleotide sequence for example.

“AAV” is an abbreviation for adeno-associated virus, and may be used to refer to the virus itself or modifications, derivatives, or pseudotypes thereof. The term covers all subtypes and both naturally occurring and recombinant forms, except where required otherwise. The abbreviation “rAAV” refers to recombinant adeno-associated virus. The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), avian AAV, bovine AAV, canine AAV, equine AAV, primate AAV, non-primate AAV, and ovine AAV, and modifications, derivatives, or pseudotypes thereof.

“Recombinant”, as applied to an AAV particle means that the AAV particle is the product of one or more procedures that result in an AAV particle construct that is distinct from an AAV particle in nature.

A recombinant adeno-associated virus particle “rAAV particle” refers to a viral particle composed of at least one AAV capsid protein and an encapsidated polynucleotide rAAV vector genome comprising a heterologous polynucleotide (i.e. a polynucleotide other than a wild-type AAV genome such as a transgene to be delivered to a mammalian cell, e.g., a transgene encoding a microdystrophin comprising the amino acid sequence of SEQ ID NO: 27). The rAAV particle may be of any AAV serotype, including any modification, derivative or pseudotype (e.g., AAV-1, AAV-2, AAV-3, AAV-4, AAV-5, AAV-6, AAV-7, AAV-8, AAV-9, or AAV-10, or derivatives/modifications/pseudotypes thereof). Such AAV serotypes and derivatives/modifications/pseudotypes, and methods of producing such serotypes/derivatives/modifications/pseudotypes are known in the art (see, e.g., Asokan et al., Mol. Ther. 20 (4): 699-708 (2012). Recombinant AAV particles comprising a transgene encoding a microdystrophin are disclosed in Int'l. Appl. Pub. No. WO 2021108755, which is incorporated herein by reference for all purposes.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where embodiments of the disclosure are described in terms of a Markush group or other grouping of alternatives, the disclosed method encompasses not only the entire group listed as a whole, but also each member of the group individually and all possible subgroups of the main group, and also the main group absent one or more of the group members. The disclosed methods also envisage the explicit exclusion of one or more of any of the group members in the disclosed methods.

### Antibodies

In certain aspects, provided herein are isolated antibodies capable of binding to a polypeptide comprising the amino acid sequence of SEQ ID NO: 1. In some embodiments, the amino acid sequence of the polypeptide consist of SEQ ID NO: 1. In some embodiments, the antibody does not bind to a peptide consisting of the amino acid of SEQ ID NO: 2. In some embodiments, the antibody comprises an antigen-binding In some embodiments, the antibody fragment comprises a single-chain Fv (scFv), F (ab) fragment, F(ab′) 2 fragment, or an isolated VH domain.

In some embodiments, the antibody is a polyclonal antibody. In some embodiments, the antibody is a monoclonal antibody.

In some embodiments, the antibody is the pAb #7684-A, pAb #7684-B or pAb #7685 polyclonal antibody. In some embodiments, the antibody is the pAb #7684-A polyclonal antibody.

In some embodiments, the isolated antibody comprises a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
the VH CDR1, CDR2 and CDR3 and the VL CDR1, CDR2 and CDR3 of the 130D2-1 antibody, respectively, independently comprising 0, 1, 2, 3, 4 or 5 substitutions;
the VH CDR1, CDR2 and CDR3 and the VL CDR1, CDR2 and CDR3 of the 133E10-1 antibody, respectively, independently comprising 0, 1, 2, 3, 4 or 5 substitutions; or
the VH CDR1, CDR2 and CDR3 and the VL CDR1, CDR2 and CDR3 of the 75A2-1 antibody, respectively, independently comprising 0, 1, 2, 3, 4 or 5 substitutions.

In some embodiments, the isolated antibody comprises a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
the VH CDR1, CDR2 and CDR3 and the VL CDR1, CDR2 and CDR3 of the 130D2-1 antibody, respectively;
the VH CDR1, CDR2 and CDR3 and the VL CDR1, CDR2 and CDR3 of the 133E10-1 antibody, respectively; or
the VH CDR1, CDR2 and CDR3 and the VL CDR1, CDR2 and CDR3 of the 75A2-1 antibody, respectively.

In some embodiments, the VH CDR1, CDR2 and CDR3 and the VL CDR1, CDR2 and CDR3 of 130D2-1, 133E10-1 and 75A2-1 are according to Kabat.

In some embodiments, the isolated antibody comprises a variable heavy chain domain (VH) and a variable light chain domain (VL), wherein the VH and VL comprises
an amino acid sequence having at least 70%, at least 80%, at least 90%, at least 95%, at least 97% or 100% identity with the VH and VL of the 130D2-1 antibody, respectively;
an amino acid sequence having at least 70%, at least 80%, at least 90%, at least 95%, at least 97% or 100% identity with the VH and VL of the 133E10-1 antibody, respectively; or
an amino acid sequence having at least 70%, at least 80%, at least 90%, at least 95%, at least 97% or 100% identity with the VH and VL of the 75A2-1 antibody, respectively.

In some embodiments, the isolated antibody comprises a variable heavy chain domain (VH) and a variable light chain domain (VL), wherein the VH and VL comprises
the VH and VL of the 130D2-1 antibody, respectively;
the VH and VL of the 133E10-1 antibody, respectively; or
the VH and VL of the 75A2-1 antibody, respectively.

In some embodiments, the isolated antibody comprises a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the
the VH CDR1 comprises an amino acid sequence of SEQ ID NO: 8 comprising 0, 1, 2, 3, 4 or 5 substitutions;
the VH CDR2 comprises an amino acid sequence of SEQ ID NO: 9 comprising 0, 1, 2, 3, 4 or 5 substitutions;
the VH CDR3 comprises an amino acid sequence of SEQ ID NO: 10 comprising 0, 1, 2, 3, 4 or 5 substitutions;
the VL CDR1 comprises an amino acid sequence of SEQ ID NO: 11 comprising 0, 1, 2, 3, 4 or 5 substitutions;
the VL CDR2 comprises an amino acid sequence of SEQ ID NO: 12 comprising 0, 1, 2, 3, 4 or 5 substitutions; and
the VL CDR3 comprises an amino acid sequence of SEQ ID NO: 13 comprising 0, 1, 2, 3, 4 or 5 substitutions.

In some embodiments, the isolated antibody comprises a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the
the VH CDR1 comprises the amino acid sequence of SEQ ID NO: 8;
the VH CDR2 comprises the amino acid sequence of SEQ ID NO: 9;
the VH CDR3 comprises the amino acid sequence of SEQ ID NO: 10;
the VL CDR1 comprises the amino acid sequence of SEQ ID NO: 11;
the VL CDR2 comprises the amino acid sequence of SEQ ID NO: 12; and
the VL CDR3 comprises the amino acid sequence of SEQ ID NO: 13.

In some embodiments, the isolated antibody comprises a variable heavy chain domain (VH) and a variable light chain domain (VL), wherein
the VH comprises an amino acid sequence having at least 70%, at least 80%, at least 90%, at least 95%, at least 97% or 100% identity with SEQ ID NO: 4; and
the VL comprises an amino acid sequence having at least 70%, at least 80%, at least 90%, at least 95%, at least 97% or 100% identity with SEQ ID NO: 5.

In some embodiments, the isolated antibody comprises a variable heavy chain domain (VH) and a variable light chain domain (VL), wherein
the VH comprises the amino acid sequence of SEQ ID NO: 4; and
the VL comprises the amino acid sequence of SEQ ID NO: 5.

In some embodiments, the isolated antibody is capable of binding to a polypeptide comprising the amino acid sequence of SEQ ID NO: 1. In some embodiments, the amino acid sequence of the polypeptide consist of SEQ ID NO: 1. In some embodiments, the antibody does not bind to a peptide consisting of the amino acid of SEQ ID NO: 2.

In certain aspects, provided herein are isolated antibodies capable of binding to a polypeptide comprising the amino acid sequence of SEQ ID NO: 3. In some embodiments, the amino acid sequence of the polypeptide consist of SEQ ID NO: 3. In some embodiments, the antibody comprises an antigen-binding In some embodiments, the antibody fragment comprises a single-chain Fv (scFv), F (ab) fragment, F(ab′) 2 fragment, or an isolated VH domain.

In some embodiments, the antibody is a polyclonal antibody. In some embodiments, the antibody is a monoclonal antibody.

In some embodiments, the isolated antibody comprises a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
the VH CDR1, 2 and 3 and the VL CDR1, CDR2 and CDR3 of the 112E4-1 antibody, respectively, independently comprising 0, 1, 2, 3, 4 or 5 substitutions;
the VH CDR1, 2 and 3 and the VL CDR1, CDR2 and CDR3 of the 115G6-1 antibody, respectively, independently comprising 0, 1, 2, 3, 4 or 5 substitutions;
the VH CDR1, 2 and 3 and the VL CDR1, CDR2 and CDR3 of the 119H2-1 antibody, respectively, independently comprising 0, 1, 2, 3, 4 or 5 substitutions;
the VH CDR1, 2 and 3 and the VL CDR1, CDR2 and CDR3 of the 121F10-1 antibody, respectively, independently comprising 0, 1, 2, 3, 4 or 5 substitutions; or
the VH CDR1, 2 and 3 and the VL CDR1, CDR2 and CDR3 of the 133D7-1 antibody, respectively, independently comprising 0, 1, 2, 3, 4 or 5 substitutions.

In some embodiments, the isolated antibody comprises a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
the VH CDR1, CDR2 and CDR3 and the VL CDR1, CDR2 and CDR3 of the 112E4-1 antibody, respectively;
the VH CDR1, CDR2 and CDR3 and the VL CDR1, CDR2 and CDR3 of the 115G6-1 antibody, respectively;
the VH CDR1, CDR2 and CDR3 and the VL CDR1, CDR2 and CDR3 of the 119H2-1 antibody, respectively;
the VH CDR1, CDR2 and CDR3 and the VL CDR1, CDR2 and CDR3 of the 121F10-1 antibody, respectively; or
the VH CDR1, CDR2 and CDR3 and the VL CDR1, CDR2 and CDR3 of the 133D7-1 antibody, respectively.

In some embodiments, the VH CDR1, CDR2 and CDR3 and the VL CDR1, CDR2 and CDR3 of 112E4-1, 115G6-1, 119H2-1, 121F10-1 and 133D7-1 are according to Kabat.

In some embodiments, the isolated antibody comprises a variable heavy chain domain (VH) and a variable light chain domain (VL), wherein the VH and VL comprises
an amino acid sequence having at least 70%, at least 80%, at least 90%, at least 95%, at least 97% or 100% identity with the VH and VL of the 112E4-1 antibody, respectively;
an amino acid sequence having at least 70%, at least 80%, at least 90%, at least 95%, at least 97% or 100% identity with the VH and VL of the 115G6-1 antibody, respectively;
an amino acid sequence having at least 70%, at least 80%, at least 90%, at least 95%, at least 97% or 100% identity with the VH and VL of the 119H2-1 antibody, respectively;
an amino acid sequence having at least 70%, at least 80%, at least 90%, at least 95%, at least 97% or 100% identity with the VH and VL of the 121F10-1 antibody, respectively; or
an amino acid sequence having at least 70%, at least 80%, at least 90%, at least 95%, at least 97% or 100% identity with the VH and VL of the 133D7-1 antibody, respectively.

In some embodiments, the isolated antibody comprises a variable heavy chain domain (VH) and a variable light chain domain (VL), wherein the VH and VL comprises
the VH and VL of the 112E4-1 antibody, respectively;
the VH and VL of the 115G6-1 antibody, respectively;
the VH and VL of the 119H2-1 antibody, respectively;
the VH and VL of the 121F10-1 antibody, respectively; or
the VH and VL of the 133D7-1 antibody, respectively.

In some embodiments, the isolated antibody comprises a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the
the VH CDR1 comprises an amino acid sequence of SEQ ID NO: 18 comprising 0, 1, 2, 3, 4 or 5 substitutions;
the VH CDR2 comprises an amino acid sequence of SEQ ID NO: 19 comprising 0, 1, 2, 3, 4 or 5 substitutions;
the VH CDR3 comprises an amino acid sequence of SEQ ID NO: 20 comprising 0, 1, 2, 3, 4 or 5 substitutions;
the VL CDR1 comprises an amino acid sequence of SEQ ID NO: 21 comprising 0, 1, 2, 3, 4 or 5 substitutions;
the VL CDR2 comprises an amino acid sequence of SEQ ID NO: 22 comprising 0, 1, 2, 3, 4 or 5 substitutions; and
the VL CDR3 comprises an amino acid sequence of SEQ ID NO: 23 comprising 0, 1, 2, 3, 4 or 5 substitutions.

In some embodiments, the isolated antibody comprises a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the
the VH CDR1 comprises the amino acid sequence of SEQ ID NO: 18;
the VH CDR2 comprises the amino acid sequence of SEQ ID NO: 19;
the VH CDR3 comprises the amino acid sequence of SEQ ID NO: 20;
the VL CDR1 comprises the amino acid sequence of SEQ ID NO: 21;
the VL CDR2 comprises the amino acid sequence of SEQ ID NO: 22; and the VL CDR3 comprises the amino acid sequence of SEQ ID NO: 23.

In some embodiments, the isolated antibody comprises a variable heavy chain domain (VH) and a variable light chain domain (VL), wherein
the VH comprises an amino acid sequence having at least 70%, at least 80%, at least 90%, at least 95%, at least 97% or 100% identity with SEQ ID NO: 14; and
the VL comprises an amino acid sequence having at least 70%, at least 80%, at least 90%, at least 95%, at least 97% or 100% identity with SEQ ID NO: 15.

An isolated antibody or antigen binding fragment thereof comprising a variable heavy chain domain (VH) and a variable light chain domain (VL), wherein
the VH comprises the amino acid sequence of SEQ ID NO: 14; and
the VL comprises the amino acid sequence of SEQ ID NO: 15.

In some embodiments, the isolated antibody is capable of binding to a polypeptide comprising the amino acid sequence of SEQ ID NO: 3. In some embodiments, the amino acid sequence of the polypeptide consist of SEQ ID NO: 3.

In some embodiments, the antibody comprises an antigen-binding In some embodiments, the antibody fragment comprises a single-chain Fv (scFv), F (ab) fragment, F(ab′) 2 fragment, or an isolated VH domain.

In some embodiments, an isolated monoclonal antibody described herein further comprises heavy and/or light chain constant regions.

In some embodiments, an isolated monoclonal antibody described herein further comprises human heavy and/or light chain constant regions.

In some embodiments, the heavy chain constant region is selected from the group consisting of human immunoglobulins IgG1, IgG2, IgG3, IgG4, IgA1, and IgA2.

In some embodiments, the heavy chain constant region comprises a native amino acid sequence.

In some embodiments, the heavy chain constant region comprises a non-native variant amino acid sequence.

In one embodiment, an antibody described herein is a recombinant antibody, a chimeric antibody, a bispecific antibody, a trispecific antibody, or a multispecific antibody. In one embodiment, the antibody fragment comprises a single-chain Fv (scFv), F (ab) fragment, F(ab′) 2 fragment, or an isolated VH domain.

In some embodiments, an antibody described herein is a multispecific antibody, e.g. a bispecific antibody. Multispecific antibodies are monoclonal antibodies that have binding specificities for at least two different sites. In some embodiments, one of the binding specificities is for a peptide having the sequence of SEQ ID NO:1 and the other is for peptide having the sequence of SEQ ID NO: 3. Bispecific antibodies can be prepared as full-length antibodies or antibody fragments.

Techniques for making multispecific antibodies, e.g., bispecific antibodies include, but are not limited to, recombinant co-expression of two immunoglobulin heavy chain-light chain pairs having different specificities (see Milstein and Cuello, Nature 305:537 (1983)), WO 93/08829, and Traunecker A., et al., EMBO J. 10:3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229:81 (1985)); using leucine zippers to produce bispecific antibodies (see, e.g., Kostelny et al., J. Immunol., 148 (5): 1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147:60 (1991). Engineered antibodies with three or more functional antigen-binding sites, including “Octopus antibodies” and dual variable domain (DVD) immunoglobulins are also included herein (see, e.g. US 2006/0025576A1 and U.S. Pat. No. 10,093,733). The antibody or fragment disclosed herein also includes a “Dual Acting Fab” or “DAF” comprising an antigen-binding site that binds to different epitopes.

In one embodiment, an antibody described herein comprises a heavy and/or light chain constant region. In one embodiment, an antibody described herein comprises a human heavy and/or light chain constant region. In one embodiment, the heavy chain constant region is human immunoglobulin IgG1, IgG2, IgG3, IgG4, IgA1, or IgA2 constant region. In one embodiment, the heavy chain constant region is human immunoglobulin IgG1 constant region. In one embodiment, the heavy chain constant region comprises a native amino acid sequence.

In another aspect, provided herein are antibodies that bind the same or an overlapping epitope of a peptide having the sequence of SEQ ID NO: 1 or 3 as an antibody described herein (e.g., 133D7-1, or 133D7-1). In certain embodiments, the epitope of an antibody can be determined by, e.g., NMR spectroscopy, X-ray crystallography, negative-stain and cryo-EM (see, e.g., Lin M, et al., J Am Soc Mass Spectrom. 5:961-971 (2018); Rantalainen et al., Cell Rep. 23 (11); 3249-3261 (2018); Torrents de la Peña A et al., PLOS Pathog. 15;15 (7): e1007920 (2019)), ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R, et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303). Antibody: antigen crystals may be studied using well-known X-ray diffraction techniques and may be refined using computer software such as Phenix (Adams et al., Acta Crystallogr Biol Crystallogr D66, 213-221 (2010)) and BUSTER (Bricogne G (1993) Acta Crystallogr D Biol Crystallogr 49 (Pt 1): 37-60; Bricogne G (1997) Meth. Enzymol. 276A: 361-423, ed Carter C W; Roversi P et al., (2000) Acta Crystallogr. D Biol. Crystallogr. 56 (Pt 10): 1316-1323). Mutagenesis mapping studies may be accomplished using any method known to one of skill in the art. See, e.g., Champe M et al., (1995) supra and Cunningham B C & Wells J A (1989) supra for a description of mutagenesis techniques, including alanine scanning mutagenesis techniques. In a specific embodiment, the epitope of an antibody is determined using alanine scanning mutagenesis studies. Usually, binding to the antigen is reduced or disrupted when a residue within the epitope is substituted to alanine. In one embodiment, the K_{D} of binding to the antigen is increased by about 5-fold, 10-fold, 20-fold, 10-fold or more when a residue within the epitope is substituted for alanine. In one embodiment, binding affinity is determined by ELISA. In addition, antibodies that recognize and bind to the same or overlapping epitopes can be identified using routine techniques such as an immunoassay, for example, by showing the ability of one antibody to block the binding of another antibody to a target antigen, i.e., a competitive binding assay.

In one embodiment, an antibody described herein immunospecifically binds to an epitope that overlaps the epitope bound by 133D7-1, or 133D7-1.

In some embodiments, an antibody described herein is capable of competing with 133D7-1, or 133D7-1 for binding to a peptide having the sequence of SEQ ID NO: 1 or 3, respectively.

In certain embodiments, the epitope of an antibody described herein is used as an immunogen to produce antibodies.

In one aspect, provided herein are methods for producing an engineered variant of an antibody described herein. In some embodiments, a method for producing an engineered variant comprises directed-evolution and yeast display. Methods for producing an engineered antibody are known to those skilled in the art, for example, as described in PCT/US2019/43578, filed on Jul. 26, 2019, which is incorporated herein by reference in its entirety for all purposes. In some embodiments, an engineered antibody possesses one or more improved properties, for example, higher binding affinity to target antigen compared to the parent antibody.

In some embodiments, a method of producing an engineered variant of a parent antibody comprises substituting one or more amino acid residues of the VH; and/or substituting one or more amino acid residues of the VL to create an engineered variant antibody, and producing the engineered variant antibody. In some embodiments, the parent antibody is an antibody described herein. In some embodiments, the parent antibody is 130D2-1, or 133D7-1. In some embodiments, the method further comprises determining that the engineered variant antibody has improved properties, for example, by determining the engineered variant antibody's binding affinity to target antigen compared to the parent antibody.

The affinity or avidity of an antibody for an antigen can be determined experimentally using any suitable method well-known in the art, e.g., flow cytometry, enzyme-linked immunoabsorbent assay (ELISA), biolayer interferometry (BLI) assay, radioimmunoassay (RIA), or kinetics (e.g., BIACORE™ analysis). Direct binding assays as well as competitive binding assay formats can be readily employed. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., K_{D} or Kd, Kₒₙ, K_{off}) are made with standardized solutions of antibody and antigen, and a standardized buffer, as known in the art and such as the buffer described herein.

In some embodiments, a peptide-specific antibody described herein is a monoclonal antibody. Monoclonal antibodies can be prepared using hybridoma methods, such as those described by Kohler and Milstein (1975) Nature 256:495. Using the hybridoma method, a host (e.g., mouse) is immunized to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g., radioimmunoassay (RIA); enzyme-linked immunosorbent assay (ELISA)) can then be propagated either in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid using any method known in the art.

In some embodiments, an antibody described herein is a monoclonal antibody. Monoclonal antibodies can be made using recombinant DNA methods, for example, as described in U.S. Pat. No. 4,816,567. The polynucleotides encoding a monoclonal antibody can be amplified from a suitable source or chemically synthetized. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells.

The polynucleotide(s) encoding a monoclonal antibody can be modified in a number of different manners using recombinant DNA technology to generate alternative antibodies. In some embodiments, the constant domains of the light and heavy chains can be substituted for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

Methods for engineering antibodies can also be used and are well-known in the art. An engineered antibody can have one or more amino acid residues substituted, deleted or inserted. These sequence modifications can be used to reduce, enhance or modify binding, affinity, on-rate, off-rate, avidity, specificity, or any other suitable characteristic, as known in the art. Antibodies can also be engineered to eliminate development liabilities by altering or eliminating sequence elements targeted for post-translational modification including glycosylation sites, oxidation sites, or deamination sites. In general, the CDR residues are directly and most substantially involved in influencing antibody binding. Accordingly, part or all of the CDR sequences are maintained while the variable framework and constant regions can be engineered by introducing substitutions, insertions, or deletions.

Antibodies disclosed herein can also optionally be engineered with retention of high affinity for the antigen and other favorable biological properties. To achieve this goal, engineered antibodies can be prepared by a process of analysis of the parental sequences and various conceptual engineered products using three-dimensional models of the parental and engineered sequences. Three-dimensional immunoglobulin models are commonly available and are familiar to those skilled in the art. Computer programs are available, which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

In certain embodiments an antibody fragment is provided. Various techniques are known for the production of antibody fragments. Traditionally, these fragments are derived via proteolytic digestion of intact antibodies (for example Morimoto et al., 1993, Journal of Biochemical and Biophysical Methods 24:107-117; Brennan et al., 1985, Science, 229:81). In certain embodiments, antibody fragments are produced recombinantly. Fab, Fv, and scFv antibody fragments can all be expressed in and secreted from E. coli or other host cells, thus allowing the production of large amounts of these fragments. Such antibody fragments can also be isolated from antibody phage libraries. The antibody fragment can also be linear antibodies as described in U.S. Pat. No. 5,641,870, for example, and can be monospecific or bispecific. Other techniques for the production of antibody fragments will be apparent to the skilled practitioner.

In certain embodiments, the variable domains in both the heavy and light chains are altered by at least partial replacement of one or more CDRs and, if necessary, by partial framework region replacement and sequence changing. Although the CDRs can be derived from an antibody of the same class or even subclass as the antibody from which the framework regions are derived, it is envisaged that the CDRs may be derived from an antibody of different class and in certain embodiments from an antibody from a different species. It may not be necessary to replace all of the CDRs with the complete CDRs from the donor variable region to transfer the antigen-binding capacity of one variable domain to another. Rather, it may only be necessary to transfer those residues that are necessary to maintain the activity of the antigen-binding site. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

The present invention further embraces variants and equivalents, which are substantially homologous to the antibodies, or antibody fragments thereof, set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well-known in the art.

In certain aspects, provided herein are compositions comprising an antibody disclosed herein. In some embodiments, the composition is a pharmaceutical composition further comprising a pharmaceutically acceptable excipient. In some embodiments, the antibody comprises an antigen-binding antibody fragment.

In certain aspects, provided herein affinity resins comprising an antibody disclosed herein and a solid support. In some embodiments, the solid support comprises a bead, gelatin, or agarose. In some embodiments, the solid support comprises a magnetic bead. In some embodiments, the antibody is attached to the solid support by covalent bonding. In some embodiments, the antibody is attached to the solid support by non-covalent association. In some embodiments, the antibody comprises an antigen-binding antibody fragment.

### Polynucleotides

In certain aspects, provided herein are polynucleotides comprising a nucleotide sequence or nucleotide sequences encoding an antibody described herein (e.g., a variable light chain and/or variable heavy chain region) or an antigen-binding fragment thereof and vectors, e.g., vectors comprising such polynucleotides. In one embodiment, the vectors can be used for recombinant expression of an antibody described herein in host cells (e.g., E. coli and mammalian cells). In some embodiments, the antibody comprises the 6 complementarity-determining regions (CDRs) of 130D2-1. In some embodiments, the antibody comprises the VH and VL domains of 130D2-1. In some embodiments, the antibody is 130D2-1. In some embodiments, the antibody comprises the 6 complementarity-determining regions (CDRs) of 133D7-1. In some embodiments, the antibody comprises the VH and VL domains of 133D7-1. In some embodiments, the antibody is 133D7-1. In some embodiments, the CDRs are according to Kabat. In some embodiment, the antibody comprises an antigen-binding antibody fragment.

In one aspect, provided herein are isolated polynucleotides encoding the heavy chain variable region or heavy chain of an antibody described herein.

In one aspect, provided herein are isolated polynucleotides encoding the light chain variable region or light chain of an antibody described herein.

In one aspect, provided herein are isolated polynucleotides encoding the heavy chain variable region or heavy chain of an antibody described herein and the light chain variable region or light chain of an antibody described herein.

In some embodiments, the polynucleotide encoding the VH and VL domains comprises the nucleotide sequence of SEQ ID NO: 6 and 7, respectively. In some embodiments, the polynucleotide encoding the VH and VL domains comprises the nucleotide sequence f SEQ ID NO: 16 and 17, respectively.

In specific aspects, provided herein is a polynucleotide comprising a nucleotide sequence encoding an antibody comprising a light chain and a heavy chain, e.g., a separate light chain and heavy chain. With respect to the light chain, in a specific embodiment, a polynucleotide provided herein comprises a nucleotide sequence encoding a kappa light chain. In another specific embodiment, a polynucleotide provided herein comprises a nucleotide sequence encoding a lambda light chain. In yet another specific embodiment, a polynucleotide provided herein comprises a nucleotide sequence encoding an antibody described herein comprising a human kappa light chain or a human lambda light chain. For example, human constant region sequences can be those described in U.S. Pat. No. 5,693,780.

In a particular embodiment, a polynucleotide provided herein comprises a nucleotide sequence encoding an antibody described herein, wherein the antibody comprises a heavy chain, and wherein the constant region of the heavy chain comprises the amino acid sequence of a human alpha or gamma heavy chain constant region.

In yet another specific embodiment, a polynucleotide provided herein comprises a nucleotide sequence encoding an antibody described herein, wherein the antibody comprises a VL domain and a VH domain comprising any amino acid sequences described herein, and wherein the constant regions comprise the amino acid sequences of the constant regions of a human IgA1, human IgA2, human IgG1 (e.g., allotype 1, 17, or 3), human IgG2, or human IgG4.

In yet another specific embodiment, a polynucleotide provided herein comprises a nucleotide sequence encoding an antibody described herein that is optimized, e.g., by codon/RNA optimization, replacement with heterologous signal sequences, and elimination of mRNA instability elements. Methods to generate optimized nucleic acids encoding an antibody that binds to a peptide having the sequence of SEQ ID NO: 1 or 3 or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid.

The polynucleotides can be obtained, and the nucleotide sequence of the polynucleotides determined, by any method known in the art. Nucleotide sequences encoding antibodies described herein, and modified versions of these antibodies can be determined using methods well-known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17:242-246), which, briefly, involves the synthesis of overlapping oligonucleotides containing portions of the sequence encoding the antibody, annealing and ligating of those oligonucleotides, and then amplification of the ligated oligonucleotides by PCR.

If a clone containing a nucleic acid encoding a particular antibody or fragment thereof is not available, but the sequence of the antibody molecule or fragment thereof is known, a nucleic acid encoding the immunoglobulin or fragment can be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well-known in the art.

In certain aspects, provided herein are cells (e.g., host cells) expressing (e.g., recombinantly) antibodies described herein, and related polynucleotides and expression vectors. Provided herein are vectors (e.g., expression vectors) comprising polynucleotides comprising nucleotide sequences encoding antibodies or an antigen-binding fragment thereof described herein. In one embodiment, the vectors can be used for recombinant expression of an antibody described herein in host cells (e.g., mammalian cells). Also provided herein are host cells comprising such vectors for recombinantly expressing antibodies described herein. In a particular aspect, provided herein are methods for producing an antibody described herein, comprising expressing such antibody in a host cell. In some embodiments, the antibody comprises the 6 complementarity-determining regions (CDRs) of 130D2-1. In some embodiments, the antibody comprises the VH and VL domains of 130D2-1. In some embodiments, the antibody is 130D2-1. In some embodiments, the antibody comprises the 6 complementarity-determining regions (CDRs) of 133D7-1. In some embodiments, the antibody comprises the VH and VL domains of 133D7-1. In some embodiments, the antibody is 133D7-1. In some embodiments, the CDRs are according to Kabat. In some embodiment, the antibody comprises an antigen-binding antibody fragment.

In certain aspects, provided herein is an isolated vector comprising a polynucleotide described herein.

In certain aspects, provided herein is a host cell comprising a polynucleotide described herein, or a vector described herein. In one embodiment, the vector encodes an antibody described herein. In one embodiment, a vector described herein comprises a first vector encoding a VH described herein and a second vector encoding a VL described herein. In one embodiment, a vector described herein comprises a first nucleotide sequence encoding a VH described herein and a second nucleotide sequence encoding a VL described herein. In some embodiments, the VH and VL comprises the amino acid sequence of SEQ ID NO: 4 and 5. In some embodiments, the VH and VL comprises the amino acid sequence of SEQ ID NO: 14 and 15. In some embodiments, the polynucleotide encoding the VH and VL domains comprises the nucleotide sequence f SEQ ID NO: 6 and 7, respectively. In some embodiments, the polynucleotide encoding the VH and VL domains comprises the nucleotide sequence f SEQ ID NO: 16 and 17, respectively.

In one embodiment, the host cell is selected from the group consisting of E. coli, Pseudomonas, Bacillus, Streptomyces, yeast, CHO, YB/20, NSO, PER-C6, HEK-293T, NIH-3T3, Helga, BHK, Hep G2, SP2/0, R1.1, B-W, L-M, COS 1, COS 7, BSC1, BSC40, BMT10 cell, plant cell, insect cell, and human cell in tissue culture. In one embodiment, the host cell is CHO.

In certain aspects, provided herein is a method of producing an antibody described herein (e.g., 130D2-1 and 133D7-1) comprising culturing a host cell described herein so that the polynucleotide is expressed and the antibody is produced. In one embodiment, the method further comprises recovering the antibody.

The isolated polypeptides, i.e., antibodies described herein can be produced by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585.

In some embodiments, a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular isolated polypeptide of interest will be inserted into an expression vector and operatively linked to an expression control sequence appropriate for expression of the protein in a desired host. Proper assembly can be confirmed by nucleotide sequencing, restriction mapping, and expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding antibodies or fragments thereof. Recombinant expression vectors are replicable DNA constructs, which have synthetic or cDNA-derived DNA fragments encoding a polypeptide chain of an antibody or fragment thereof operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor, which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A variety of host-expression vector systems can be utilized to express antibody molecules described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NSO, PER.C6, VERO, CRL7030, HsS78Bst, Helga, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies described herein are CHO cells, for example CHO cells from the CHO GS System™ (Lonza). In a particular embodiment, cells for expressing antibodies described herein are human cells, e.g., human cell lines. In a specific embodiment, a mammalian expression vector is pOptiVEC™ or pcDNA3.3. In a particular embodiment, bacterial cells such as E. coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45:101-105; and Cockett M I et al., (1990) Biotechnology 8:662-667). In certain embodiments, antibodies described herein are produced by CHO cells or NSO cells. In a specific embodiment, the expression of nucleotide sequences encoding antibodies described herein is regulated by a constitutive promoter, inducible promoter or tissue specific promoter.

Suitable host cells for expression of a polypeptide of interest such as an antibody described herein include prokaryotes, yeast, insect or higher eukaryotic cells under the control of appropriate promoters. Prokaryotes include gram negative or gram-positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference. Additional information regarding methods of protein production, including antibody production, can be found, e.g., in U.S. Patent Publication No. 2008/0187954, U.S. Pat. Nos. 6,413,746 and 6,660,501, and International Patent Publication No. WO 04009823, each of which is hereby incorporated by reference herein in its entirety.

Various mammalian or insect cell culture systems are also advantageously employed to express a recombinant protein such as an antibody described herein. Expression of recombinant proteins in mammalian cells can be performed because such proteins are generally correctly folded, appropriately modified and completely functional. Examples of suitable mammalian host cell lines include but are not limited to CHO, VERO, BHK, Hela, MDCK, HEK 293, NIH 3T3, W138, BT483, Hs578T, HTB2, BT20 and T47D, NSO (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza HA peptide sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography.

For example, supernatants from systems, which secrete recombinant protein, e.g., an antibody, into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further an agent. Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005, each of which is hereby incorporated by reference herein in its entirety.

In specific embodiments, an antibody described herein is isolated or purified. Generally, an isolated antibody is one that is substantially free of other antibodies with different antigenic specificities than the isolated antibody. For example, in a particular embodiment, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody. When the polypeptide (e.g., antibody described herein) is recombinantly produced, it is also generally substantially free of culture medium, i.e., culture medium represents less than about 20%, 10%, 2%, 1%, 0.5%, or 0.1% of the volume of the protein preparation. When the polypeptide (e.g., antibody described herein) is produced by chemical synthesis, it is generally substantially free of chemical precursors or other chemicals, i.e., it is separated from chemical precursors or other chemicals, which are involved in the synthesis of the protein. Accordingly, such preparations of the polypeptide (e.g., antibody described herein) have less than about 30%, 20%, 10%, or 5% (by dry weight) of chemical precursors or compounds other than the polypeptide of interest. In one embodiment, antibodies described herein are isolated or purified.

### Methods for Detecting, Isolating or Quantifying a Peptide or Protein

In certain aspects, provided herein are methods for detecting, isolating or quantifying a peptide in a sample comprising contacting the peptide with an antibody disclosed herein, wherein the amino acid sequence of the peptide comprises SEQ ID NO: 1 or 3. In some embodiments, the amino acid sequence of the peptide comprises SEQ ID NO: 1. In some embodiments, the amino acid sequence of the peptide comprises SEQ ID NO: 3. In some embodiments the sample comprises a protease digested protein isolate. In some embodiments, the protein isolate was obtained from a subject, for example, from a skeletal muscle tissue of the subject. In some embodiments, the protease is trypsin. In some embodiments, the sample comprises a peptide comprising SEQ ID NO: 1 and a peptide comprising SEQ ID NO: 3.

In certain aspects, provided herein are methods for detecting or quantifying a recombinant polypeptide in a sample comprising a protease, e.g., trypsin digested protein isolate, wherein the method comprises contacting the peptide with an antibody disclosed herein, wherein the amino acid sequence of the recombinant polypeptide comprises SEQ ID NO: 28 and/or 29.

In certain aspects, provided herein are methods for detecting or quantifying dystrophin and/or microdystrophin expression in a subject. In some embodiments, the method comprises contacting a sample with an antibody disclosed herein, wherein the sample comprises a protease, e.g., trypsin digested protein isolate. In some embodiments, dystrophin is a full-length dystrophin. In some embodiments, dystrophin is endogenous dystrophin. In some embodiments, dystrophin is recombinant dystrophin. In some embodiments, dystrophin is a mutated or engineered dystrophin. In some embodiments, dystrophin is a mutated or engineered dystrophin that binds the anti-LLQ mAb, but does not bind to the anti-LEM mAb. In some embodiments, the microdystrophin binds the anti-LLQ mAb, but does not bind to the anti-LEM mAb. In some embodiments, the microdystrophin has an amino acid sequence comprising SEQ ID NO: 27. In some embodiments, the sample comprises a protease, e.g., trypsin digested protein isolate from a subject who has been administered a recombinant polynucleotide encoding the microdystrophin, e.g., a recombinant adeno-associated virus comprising the polynucleotide encoding the microdystrophin. In some embodiments, the sample comprises protease (e.g., trypsin) digested microdystrophin and protease digested dystrophin, wherein the protease digestion of microdystrophin releases the LEM peptide of SEQ ID NO: 1. In some embodiments, the method described herein is used to simultaneously detect or quantify microdystrophin and dystrophin in a sample.

In some embodiments, provided herein are methods of isolating a peptide comprising the amino acid sequence of SEQ ID NO: 1 or 3 from a sample via immunoaffinity purification, ligand binding assay (LBA), or equivalent assay. In some embodiments, the method of isolating the peptide from a sample comprises (a) contacting the sample comprising the peptide with a composition comprising the antibody described herein under conditions that permit binding of the peptide to the antibody, (b) removing a portion of the sample that is not bound to the antibody; and (c) dissociating the peptide from the antibody. In some embodiments, the composition comprising the antibody comprises an affinity resin comprising a solid support and an antibody described herein. In some embodiments, the solid support is selected from the group consisting of a bead, gelatin, or agarose. In some embodiments, the solid support is a magnetic bead. In some embodiments, the antibody is attached to the solid support by covalent bonding. In some embodiments, the antibody is attached to the solid support by non-covalent association. In some embodiments, the antibody comprises a tag, for example, biotin, hexa-histidine tag or FLAG-tag to facilitate the purification of the peptide. In some embodiments, the sample comprises a peptide comprising the amino acid sequence of SEQ ID NO: 1 and a peptide comprising the amino acid sequence of SEQ ID NO: 3. In some embodiments, the method described herein is used to simultaneously isolate the peptide comprising the amino acid sequence of SEQ ID NO: 1 and the peptide comprising the amino acid sequence of SEQ ID NO: 3.

In some embodiments of a method of isolating a peptide comprising the amino acid sequence of SEQ ID NO: 1 described herein, the antibody comprises 6 CDRs comprising the amino acid sequence of SEQ ID NO: 8-12 and 13. In some embodiments, the antibody comprises the VH and VL of SEQ ID NO: 4 and 5, respectively.

In some embodiments of a method of isolating a peptide comprising the amino acid sequence of SEQ ID NO: 3 described herein, the antibody comprises 6 CDRs comprising the amino acid sequence of SEQ ID NO: 18-22 and 23. In some embodiments, the antibody comprises the VH and VL of SEQ ID NO: 14 and 15, respectively.

In some embodiments of a method of isolating a peptide comprising the amino acid sequence of SEQ ID NO: 1 or 3 described herein, the antibody is a polyclonal antibody described herein, e.g., pAb #7684-A.

In some embodiments, provided herein are methods of quantifying a peptide comprising the amino acid sequence of SEQ ID NO: 1 or 3 in a sample comprising contacting the peptide with an antibody described herein. In some embodiments, the method comprises a radioimmunoassay, immunoaffinity (IA) assay, or ligand-binding assay (LBA). In some embodiments, the method comprises isolating the peptide from the sample and determining the amount of peptide recovered. In some embodiments, the method comprises (a) contacting the sample comprising the peptide with a composition comprising the antibody described herein under conditions that permit binding of the peptide to the antibody, (b) removing a portion of the sample that is not bound to the antibody; (c) recovering the peptide; and (d) determining the amount of peptide recovered in step (c). In some embodiments, the amount of peptide is determined by LC/MS or LC-MS/MS. In some embodiments, the sample further comprises a stable isotope labeled peptide standard comprising the amino acid sequence of SEQ ID NO: 1 or 3. In some embodiments, the method provides absolute quantification of the peptide. In some embodiments, the method provides relative quantification of the peptide. In some embodiments, the antibody is a monoclonal antibody disclosed herein (e.g., 130D2-1 or 133D7-1). In some embodiments, the antibody is a polyclonal antibody disclosed herein, e.g., pAb #7684-A. In some embodiments, the method described herein is used to simultaneously quantify a peptide comprising the amino acid sequence of SEQ ID NO: 1 and a peptide comprising the amino acid sequence of SEQ ID NO: 3 in a sample.

In some embodiments, the sample comprises a protease digested protein isolate obtained from a subject. In some embodiments, the sample comprises a protease digested protein isolate obtained from a skeletal muscle tissue of the subject. In some embodiments, the skeletal muscle is gastrocnemius, quadriceps or diaphragm. In some embodiments, the sample comprises a protease digested protein isolate obtained from the heart muscle of the subject. In some embodiments, the protease comprises trypsin. In some embodiments, the subject is a human, primate or murine subject. In some embodiments, the subject has been administered a recombinant polypeptide comprising the amino acid sequence of SEQ ID NO: 28 and/or 29. In some embodiments, the subject has been administered a recombinant polynucleotide encoding a recombinant polypeptide comprising the amino acid sequence of SEQ ID NO: 28 and/or 29. In some embodiments, the subject has been administered a recombinant virus comprising a polynucleotide encoding a recombinant polypeptide comprising the amino acid sequence of SEQ ID NO: 28 and/or 29. In some embodiments, the recombinant virus is a recombinant adeno-associated virus. In some embodiments, the recombinant polypeptide is microdystrophin. In some embodiments, the microdystrophin comprises the amino acid sequence of SEQ ID NO: 27.

In some embodiments, provided herein are methods for detecting or quantifying a recombinant polypeptide in a sample, wherein the amino acid sequence of the recombinant polypeptide comprises SEQ ID NO: 28 and/or 29. A skilled artisan understands that protease digestion, e.g., trypsin digestion, of a polypeptide having an amino acid sequence comprising SEQ ID NO: 28 and 29 releases a peptide of SEQ ID NO: 1 and 3, respectively. A skilled artisan further understands that methods described herein for detecting or quantifying a peptide having the amino acid sequence of SEQ ID NO: 1 or 3 can also be used for detecting or quantifying a recombinant polypeptide having an amino acid sequence comprising SEQ ID NO: 28 and/or 29 based on the fact that protease digestion of the recombinant polypeptide releases a peptide of SEQ ID NO: 1 and/or 3, respectively. In some embodiments, the recombinant polypeptide is microdystrophin comprising the amino acid sequence of SEQ ID NO: 27.

In some embodiments, provided herein is a method of quantifying the level of a recombinant polypeptide in a subject comprises (a) providing a sample comprising a protease digested protein isolate obtained from the subject, wherein the sample comprises one or more peptides having the amino acid sequence of SEQ ID NO: 1 or 3; (b) contacting the sample with a composition comprising an antibody described herein under conditions that permit binding of the antibody to the peptide; (c) recovering the peptide bound to the antibody; and (d) determining the amount of peptide recovered in step (c). In some embodiments, the amount of peptide is determined by LC/MS or LC-MS/MS. In some embodiments, the sample comprises a protease digested protein isolate obtained from a skeletal muscle tissue of the subject. In some embodiments, the subject is a human, primate or murine subject. In some embodiments, the subject has been administered the recombinant polypeptide comprising the amino acid sequence of SEQ ID NO: 28 and/or 29. In some embodiments, the subject has been administered a recombinant polynucleotide encoding a recombinant polypeptide comprising the amino acid sequence of SEQ ID NO: 28 and/or 29. In some embodiments, the subject has been administered a recombinant virus comprising a polynucleotide encoding a recombinant polypeptide comprising the amino acid sequence of SEQ ID NO: 28 and/or 29. In some embodiments, the recombinant virus is a recombinant adeno-associated virus. In some embodiments, the recombinant polypeptide is microdystrophin. In some embodiments, the microdystrophin comprises the amino acid sequence of SEQ ID NO: 27. In some embodiments, the antibody is a monoclonal antibody disclosed herein (e.g., 130D2-1 or 133D7-1). In some embodiments, the antibody is a polyclonal antibody disclosed herein, e.g., pAb #7684-A.

In some embodiments, provided herein are methods for detecting or quantifying dystrophin and/or microdystrophin expression in a subject. Protease, e.g., trypsin digestion of dystrophin releases a peptide having the sequence of SEQ ID NO: 3. Protease, e.g., trypsin digestion of a microdystrophin comprising the amino acid sequence of SEQ ID NO: 27 releases peptides having the sequence of SEQ ID NO: 1 or 3. A skilled artisan understands that methods described herein for detecting or quantifying a peptide having the amino acid sequence of SEQ ID NO: 3 can be used to detect or quantify the level of dystrophin and microdystrophin in a sample following protease, e.g., trypsin digestion of the sample. A skilled artisan further understands that methods described herein for detecting or quantifying a peptide having the amino acid sequence of SEQ ID NO: 1 can be used to detect or quantify the level of microdystrophin in a sample following protease, e.g., trypsin digestion of the sample. Thus, using the methods described herein, a skilled artisan is able to monitor microdystrophin expression in a subject who has been administered gene therapy, e.g., AAV-mediated gene therapy to deliver a recombinant polynucleotide encoding the microdystrophin. In some embodiments, the method described herein is used to simultaneously detect or quantify dystrophin and microdystrophin in a sample.

In some embodiments, provided herein is a method of quantifying the level of dystrophin and/or microdystrophin expression in a subject comprising (a) providing a sample comprising a protease digested protein isolate obtained from the subject, wherein the sample comprises one or more peptides having the amino acid sequence of SEQ ID NO: 1 or 3; (b) contacting the sample with a composition comprising an antibody described herein under conditions that permit binding of the antibody to the peptide; (c) recovering the peptide bound to the antibody; and (d) determining the amount of peptide recovered in step (c), wherein the amino acid sequence of the microdystrophin comprises SEQ ID NO: 28 and/or 29. In some embodiments, the amount of peptide is determined by LC/MS or LC-MS/MS. In some embodiments, the method provides an absolute quantification of the level of dystrophin and/or microdystrophin expression. In some embodiments, the method provides a relative quantification of the level of dystrophin and/or microdystrophin expression. In some embodiments, the antibody is a monoclonal antibody disclosed herein (e.g., 130D2-1 or 133D7-1). In some embodiments, the antibody is a polyclonal antibody disclosed herein, e.g., pAb #7684-A. In some embodiments, the method described herein is used to simultaneously quantify the level of dystrophin and microdystrophin expression in the subject.

In some embodiments, provided herein is a method of quantifying the level of dystrophin and/or microdystrophin expression in a subject comprising (a) providing a sample comprising a protease digested protein isolate obtained from the subject, wherein the sample comprises one or more peptides having the amino acid sequence of SEQ ID NO: 1 or 3; (b) contacting the sample with a composition comprising a first antibody described herein that is capable of binding to the LEM peptide (SEQ ID NO: 1) under conditions that permit binding of the antibodies to its peptide target; (c) recovering the peptide bound to the antibody; and (d) determining the amount of peptide recovered in step (c). In some embodiments, provided herein is a method of quantifying the level of dystrophin and/or microdystrophin expression in a subject comprising (a) providing a sample comprising a protease digested protein isolate obtained from the subject, wherein the sample comprises one or more peptides having the amino acid sequence of SEQ ID NO: 1 or 3; (b) contacting the sample with (i) a composition comprising a first antibody described herein that is capable of binding to the LEM peptide (SEQ ID NO: 1) and (ii) a composition comprising a second antibody that is capable of binding to a protease digested dystrophin peptide under conditions that permit binding of the first and second antibodies to their respective peptide targets; (c) recovering the peptides bound to the antibodies; and (d) determining the amount of the respective peptides recovered in step (c). In some embodiments, the amino acid sequence of the microdystrophin comprises SEQ ID NO: 28 and/or 29. In some embodiments, the antibody that is capable of binding to the LEM peptide (SEQ ID NO: 1) comprises 130D2-1. In some embodiments, the antibody that is capable of binding to a protease digested dystrophin peptide is capable of binding to the LLQ peptide (SEQ ID NO: 3). In some embodiments, the antibody that is capable of binding to a protease digested dystrophin peptide is an antibody disclosed herein that is capable of binding to the LLQ peptide (SEQ ID NO: 3). In some embodiments, the antibody that is capable of binding to a protease digested dystrophin peptide comprises 133D7-1, 112E4-1, 115G6-1, or 121F10-1 antibody, or an antibody that binds to the same or an overlapping epitope of a peptide having the sequence of SEQ ID NO: 3 as the 133D7-1, 112E4-1, 115G6-1, or 121F10-1 antibody. In some embodiments, the antibody that is capable of binding to a protease digested dystrophin peptide comprises 133D7-1. In some embodiments, the antibody that is capable of binding to a protease digested dystrophin peptide also binds to a protease digested microdystrophin or engineered dystrophin peptide. Other antibodies or antigen binding fragments thereof which bind to dystrophin are known in the art. Examples include but are not limited to: ab275391 (Abcam), ab218198 (Abcam), ab15277 (Abcam), NCL-DYSB (Leica Biosystems). Additional antibodies which bind to dystrophin are disclosed at abcam.com and labome.com. In some embodiments, the amount of peptide is determined by LC/MS or LC-MS/MS. In some embodiments, the method provides an absolute quantification of the level of dystrophin and/or microdystrophin expression. In some embodiments, the method provides a relative quantification of the level of dystrophin and/or microdystrophin expression. In some embodiments, the method described herein is used to simultaneously quantify the level of dystrophin and microdystrophin expression in the subject.

In some embodiments, the sample comprises a protease digested protein isolate obtained from a skeletal muscle tissue of the subject. In some embodiments, the protease is trypsin.

In some embodiments, the subject is a human, primate or murine subject. In some embodiments, the subject is a human. In some embodiments, the subject is a primate. In some embodiments, the subject is a murine. In some embodiments, the subject suffers from Duchenne muscular dystrophy.

In some embodiments, the subject is a non-human mammal that has been genetically modified to comprise one or more mutations in the dystrophin gene.

In some embodiments, the subject has been administered a recombinant polynucleotide encoding a microdystrophin comprising the amino acid sequence of SEQ ID NO: 28 and 29. In some embodiments, the microdystrophin comprises the amino acid sequence of SEQ ID NO: 27. In some embodiments, the recombinant polynucleotide is DNA. In some embodiments, the recombinant polynucleotide is RNA. In some embodiments, the RNA is mRNA comprising a modified ribonucleotide.

In some embodiments, the subject has been administered a recombinant virus comprising a polynucleotide encoding a microdystrophin comprising the amino acid sequence of SEQ ID NO: 28 and 29. In some embodiments, the microdystrophin comprises the amino acid sequence of SEQ ID NO: 27. In some embodiments, the recombinant virus is a recombinant adeno-associated virus.

An antibody described herein can be used to detect a peptide comprising the amino acid sequence of SEQ ID NO: 1 or 3 in a sample, e.g., biological sample, using classical immunological methods known to those of skill in the art, including immunoassays, such as the enzyme linked immunosorbent assay (ELISA), immunoprecipitation, or Western blotting. In one embodiment, an antibody described herein is conjugated with a detectable label. Suitable assay labels are known in the art and include enzyme labels, such as, glucose oxidase; radioisotopes, such as iodine (125I, 1211), carbon (14C), sulfur (35S), tritium (3H), indium (121 In), and technetium (99Tc); luminescent labels, such as luminol; and fluorescent labels, such as fluorescein and rhodamine, and biotin. Such labels can be used to label an antibody described herein. Alternatively, a second antibody that recognizes an antibody described herein can be labeled and used in combination with the antibody described herein to detect a peptide comprising the amino acid sequence of SEQ ID NO: 1 or 3.

In some aspects, provided herein are methods for detecting a peptide comprising the amino acid sequence of SEQ ID NO: 1 or 3 in a sample, comprising contacting said sample with an antibody described herein. In some embodiments, an antibody described herein can carry a detectable or functional label. An antibody described herein can carry a fluorescence label. Exemplary fluorescence labels include, for example, Fluorescein, Texas red, Alexa Fluor dyes, Cy dyes and DyLight dyes. An antibody described herein can carry a radioactive label. When radioactive labels are used, currently available counting procedures known in the art may be utilized to identify and quantitate the specific binding of an antibody described herein. In the instance where the label is an enzyme, detection may be accomplished by any of the presently utilized colorimetric, spectrophotometric, fluorospectrophotometric, amperometric or gasometric techniques as known in the art. This can be achieved by contacting a sample or a control sample with an antibody described herein under conditions that allow for the formation of a complex between the antibody and a peptide comprising the amino acid sequence of SEQ ID NO: 1 or 3. Any complexes formed between the antibody and the peptide are detected and compared in the sample and the control. In some embodiments, the method described herein is used to simultaneously detect a peptide comprising the amino acid sequence of SEQ ID NO: 1 and a peptide comprising the amino acid sequence of SEQ ID NO: 3 in the sample.

### Kits

In certain aspects, provided herein are kits for detecting, isolating or quantifying a peptide in a sample, wherein the kit comprises one or more antibodies disclosed herein that is capable of binding a peptide having the amino acid sequence of SEQ ID NO: 1 or 3. In some embodiments, the kit further comprises an isolated peptide having the sequence of SEQ ID NO: 1 and/or an isolated peptide having the sequence of SEQ ID NO: 3. In some embodiments, the isolated peptide or peptides are a stable isotope labeled peptide.

In certain aspects, provided herein are kits for detecting or quantifying a recombinant polypeptide in a sample, wherein the kit comprises one or more antibodies disclosed herein that is capable of binding a peptide having the amino acid sequence of SEQ ID NO: 1 or 3, and wherein the amino acid sequence of the recombinant polypeptide comprises SEQ ID NO: 28 and/or 29. In some embodiments, the kit further comprises an isolated peptide having the sequence of SEQ ID NO: 1 and/or an isolated peptide having the sequence of SEQ ID NO: 3. In some embodiments, the kit further comprises a composition comprising the recombinant polypeptide, optionally, a stable isotope labeled recombinant polypeptide. In some embodiments, the recombinant polypeptide is microdystrophin. In some embodiments, the microdystrophin comprises the amino acid sequence of SEQ ID NO: 27. In some embodiments, the isolated peptide or peptides are a stable isotope labeled peptide.

In certain aspects, provided herein are kits for detecting or quantifying dystrophin and/or microdystrophin expression in a subject, wherein the kit comprises one or more antibodies disclosed herein that is capable of binding a peptide having the amino acid sequence of SEQ ID NO: 1 or 3, and wherein the amino acid sequence of the microdystrophin comprises SEQ ID NO: 28 and/or 29. In some embodiments, the kit is for detecting or quantifying microdystrophin expression in a subject. In some embodiments, the kit further comprises an isolated peptide having the sequence of SEQ ID NO: 1 and/or an isolated peptide having the sequence of SEQ ID NO: 3. In some embodiments, the kit is for detecting or quantifying microdystrophin expression in a subject. In some embodiments, the kit further comprises a composition comprising the microdystrophin, optionally, a stable isotope labeled microdystrophin. In some embodiments, the microdystrophin comprises the amino acid sequence of SEQ ID NO: 27. In some embodiments, the isolated peptide or peptides are a stable isotope labeled peptide.

In some embodiments, the kit comprises an antibody disclosed herein that is capable of binding a peptide having the amino acid sequence of SEQ ID NO: 1 and an isolated peptide having the amino acid sequence of SEQ ID NO: 1. In some embodiments, the antibody is 130D2-1. In some embodiments, the antibody comprises the 6 CDR sequences of 130D2-1. In some embodiments, the antibody comprises 6 CDR sequences having the amino acid sequence of SEQ ID NO: 8-12 and 13. In some embodiments, the antibody comprises the VH and VL domains of 130D2-1. In some embodiments, the antibody comprises a VH and VL comprising the amino acid sequences of SEQ ID NO: 4 and 5, respectively. In some embodiments, the peptide is a stable isotope labeled peptide.

In some embodiments, the kit comprises an antibody disclosed herein that is capable of binding a peptide having the amino acid sequence of SEQ ID NO: 3 and an isolated peptide having the amino acid sequence of SEQ ID NO: 3. In some embodiments, the antibody is 133D7-1. In some embodiments, the antibody comprises the 6 CDR sequences of 133D7-1. In some embodiments, the antibody comprises 6 CDR sequences having the amino acid sequence of SEQ ID NO: 18-22 and 23. In some embodiments, the antibody comprises the VH and VL domains of 133D7-1. In some embodiments, the antibody comprises a VH and VL comprising the amino acid sequences of SEQ ID NO: 14 and 15, respectively. In some embodiments, the peptide is a stable isotope labeled peptide.

In some embodiments, the kit comprises an antibody disclosed herein that is capable of binding a peptide having the amino acid sequence of SEQ ID NO: 1, an antibody disclosed herein that is capable of binding a peptide having the amino acid sequence of SEQ ID NO: 3, an isolated peptide having the amino acid sequence of SEQ ID NO: 1, and an isolated peptide having the amino acid sequence of SEQ ID NO: 3. In some embodiments, the antibody capable of binding a peptide having the amino acid sequence of SEQ ID NO: 1 is 130D2-1, and the antibody capable of binding a peptide having the amino acid sequence of SEQ ID NO: 3 is 133D7-1. In some embodiments, the antibody capable of binding a peptide having the amino acid sequence of SEQ ID NO: 1 comprises the 6 CDRs of 130D2-1, and the antibody capable of binding a peptide having the amino acid sequence of SEQ ID NO: 3 comprises the 6 CDRs of 133D7-1. In some embodiments, the antibody capable of binding a peptide having the amino acid sequence of SEQ ID NO: 1 comprises the VH and VL of 130D2-1, and the antibody capable of binding a peptide having the amino acid sequence of SEQ ID NO: 3 comprises the VH and VL of 133D7-1. In some embodiments, the peptides having the amino acid sequence of SEQ ID NO: 1 or 3 are stable isotope labeled peptides. In some embodiments, the kit further comprises a composition comprising a microdystrophin, optionally, a stable isotope labeled microdystrophin. In some embodiments, the microdystrophin comprises the amino acid sequence of SEQ ID NO: 27.

### EXAMPLES

### Example 1—Candidate Tryptic Peptides for the Quantification of Microdystrophin A

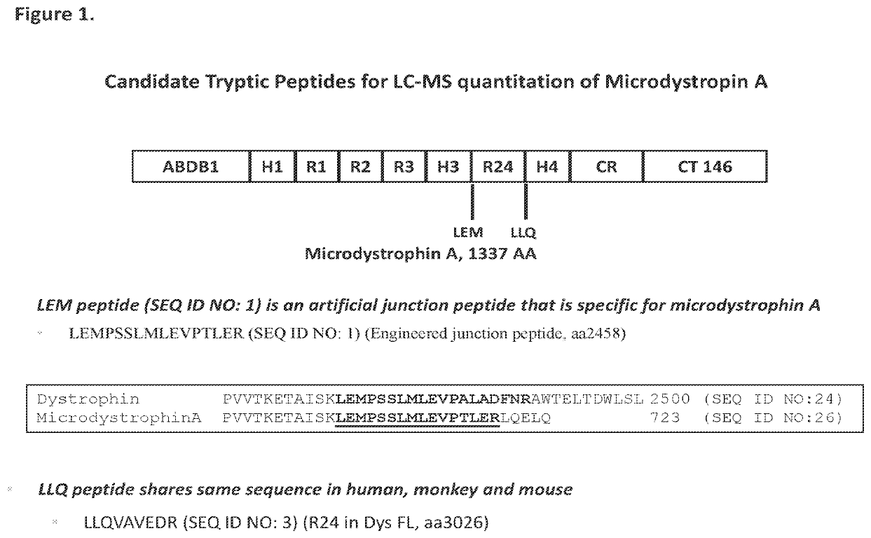

Various microdystrophin constructs, including microdystrophin A (SEQ ID NO: 27) have been developed for use in gene therapy for congenital diseases, e.g., Duchenne muscular dystrophy caused by mutations in the dystrophin gene that result in the reduction or loss of wild type dystrophin protein. See, e.g., Int'l Appl. Pub. No. WO 2021108755, which is incorporated herein by reference for all purposes. Two tryptic peptides were selected for the monitoring of microdystrophin expression. FIG. 1. The LEM peptide (SEQ ID NO: 1) encompasses an artificial junction that is specific for microdystrophin A, whereas the LLQ peptide (SEQ ID NO: 3) is present both in full-length dystrophin and in microdystrophin A. As such, detection and quantification of the LEM peptide allows the monitoring of microdystrophin A expression. On the other hand, detection and quantification of the LLQ peptide allows the monitoring of total dystrophin expression, i.e., the sum of full-length dystrophin and microdystrophin A expression. Furthermore, because the LLQ peptide sequence is present in the human, primate and murine dystrophin polypeptide, detection and quantification of the LLQ peptide allows the monitoring of total dystrophin expression in all of these organisms.

A skilled artisan further appreciates that the reagents and methods for detecting and quantifying the LEM and LLQ peptides disclosed herein can be used to monitor the expression of any polypeptide or protein whose digestion with trypsin releases the LEM and/or LLQ peptide.

### Example 2—LEM Peptide Specific Antibodies

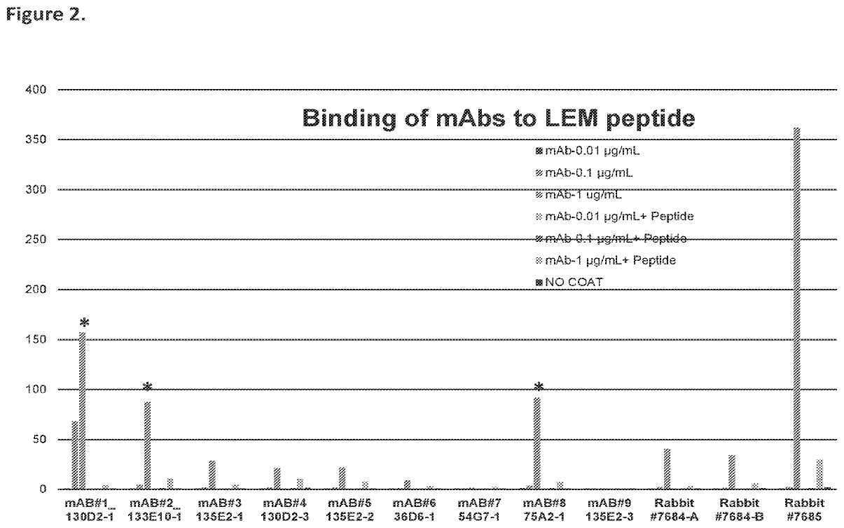

LEM peptide specific rabbit polyclonal and monoclonal antibodies were produced using standard laboratory technologies. Binding affinity of the antibodies was assessed using MSD experiment performed according to the manufacturer's protocols. MSD plates were coated with 1 mg/ml of peptide in PBS and blocked with casein in PBS. Anti-rabbit sulfo-tagged antibody was used as the detection agent. Antibody binding specificity was assessed using control reactions that included the target peptide to inhibit binding. Results are shown in FIG. 2. Binding affinity and specificity is shown in Table 1 below. Data show good binding of affinity and specificity of antibodies 130D2-1, 133E10-1 and 75A2-1 to the LEM peptide. None of the antibodies tested can bind to a fragment of the LEM peptide having the amino acid sequence of LEMPSSLMLEVP (SEQ ID NO: 2).

TABLE 1
Binding affinity and specificity of select anti-LEM
peptide antibodies

| | S/N Rate | | Inhibition |
|---|---|---|---|
| Ab # | mAb-1 ug/mL | mAb-1 μg/mL + Peptide | Rate |
| 130D2-1 | 157 | 4 | 97% |
| 133E10-1 | 88 | 11 | 87% |
| 135E2-1 | 29 | 5 | 84% |
| 75A2-1 | 92 | 8 | 92% |
| pAb# 7684-A* | 41 | 3 | 92% |
| pAb# 7684-B | 34 | 6 | 83% |
| pAb# 7685 | 362 | 30 | 92% |

### Example 3—LLQ Peptide Specific Antibodies

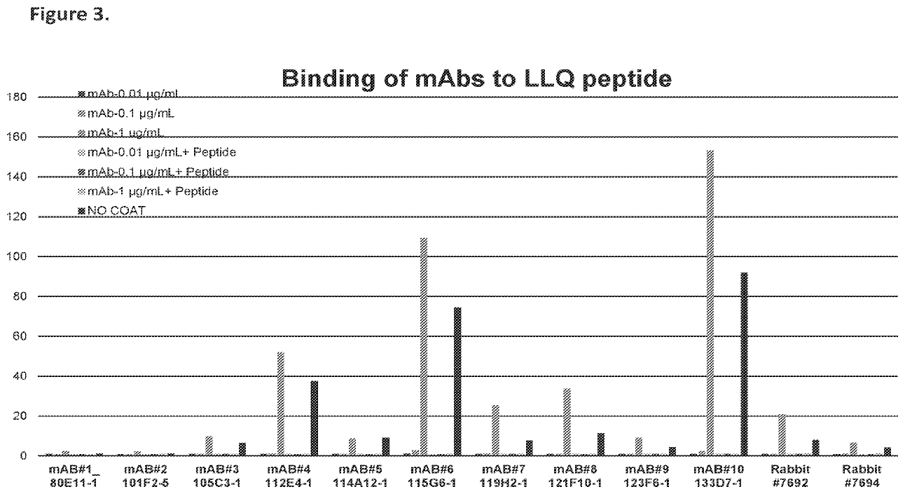

LLQ peptide specific rabbit polyclonal and monoclonal antibodies were produces using standard laboratory technologies. Binding affinity of the antibodies was assessed using MSD experiment performed according to the manufacturer's protocols. MSD plates were coated with 1 mg/ml of peptide in PBS and blocked with casein in PBS. Anti-rabbit sulfo-tagged antibody was used as the detection agent. Antibody binding specificity was assessed using control reactions that included the target peptide to inhibit binding. Results are shown in FIG. 3. Binding affinity and specificity is shown in Table 2 below. Data show that monoclonal antibodies 112E4-1, 115G6-1, 119H2-1, 121F10-1 and 133D7-1 have better binding affinity to LLQ peptide than the polyclonal antibodies (pAbs). Most anti-LLQ monoclonal antibodies (mAb) and polyclonal antibodies (pAb) bind to casein blocker, but the binding can be inhibited by LLQ peptide (data not shown).

TABLE 2
Binding affinity and specificity of select anti-LLQ
peptide antibodies

| | S/N Rate | | Inhibition |
|---|---|---|---|
| Ab # | mAb-1 ug/mL | mAb-1 μg/mL + Peptide | Rate |
| 112E4-1 | 52 | 1 | 98% |
| 115G6-1 | 109 | 1 | 99% |
| 119H2-1 | 25 | 1 | 96% |
| 121F10-1 | 34 | 1 | 97% |
| 133D7-1 | 153 | 1 | 99% |
| pAb# 7692 | 21 | 1 | 94% |
| pAb# 7694 | 6 | 1 | 81% |

### Example 4—Screening of Anti-LEM and LLQ Peptide Antibodies for Use in LCMS

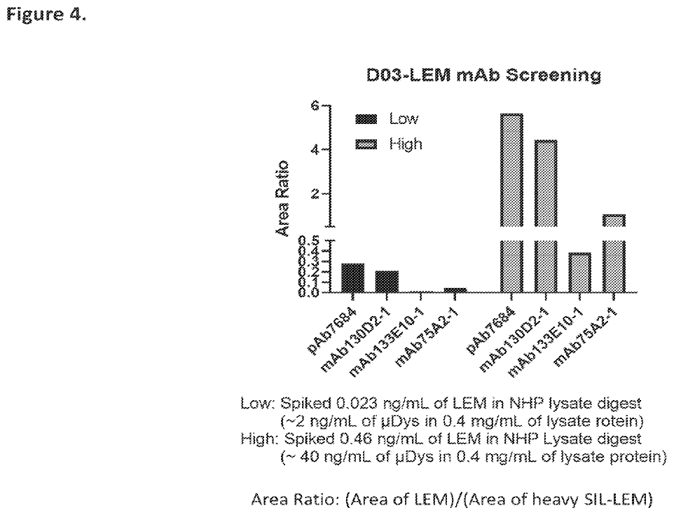

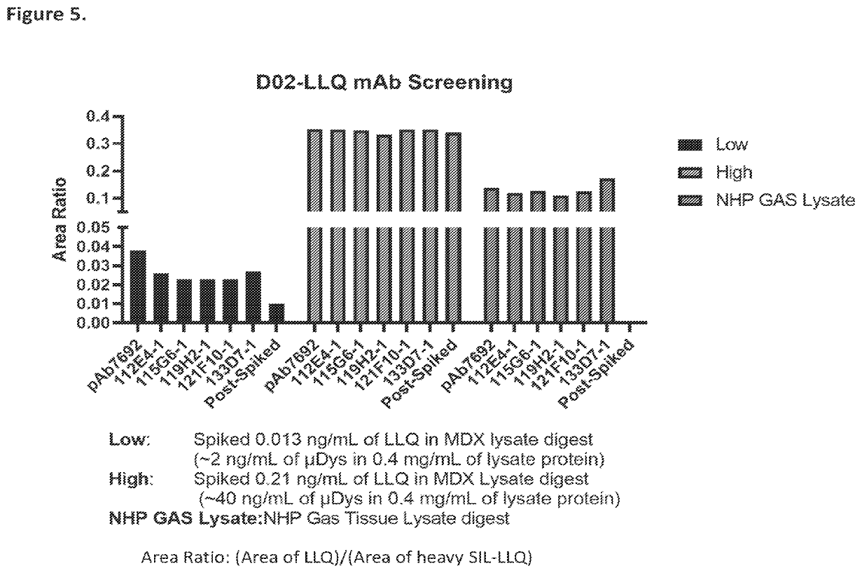

Anti-LEM and LLQ peptide antibodies were screened for use in LCMS. Briefly, diluted tissue lysate was digested by trypsin overnight. Remaining trypsin activity was quenched by adding 1% protease inhibitor. The diluted tissue lysate was spiked with synthetic LEM or LLQ peptide at low and high concentration levels. LEM peptide spike was directly added to trypsin digested non-human primate (NHP) gastrocnemius (GAS) tissue lysate. LLQ peptides spike was added to digested dystrophin defective mdx GAS tissue lysate; NHP GAS lysate was used as matrix control. Each sample was immunocaptured using immobilized polyclonal or anti-peptide IgG monoclonal antibody on Pierce protein A/G magnetic beads. Bound peptides were eluted from beads with acid. A standard amount of stable isotope labeled LEM or LLQ peptide was added to the eluted sample for quantification. LCMS results obtained using the various anti-LEM and anti-LLQ antibodies are shown in FIGS. 4 and 5, respectively. The ratio of unlabeled to stable isotope labeled peptides detected in the eluted samples is shown. The 130D2-1 monoclonal anti-LEM antibody provided comparable results with the anti-LEM polyclonal antibody at both high and low spike conditions. All 5 anti-LLQ mAbs had similar performance as the pAb, with the 133D7-1 anti-LLQ antibody showing slightly better results.

### Example 5—LCMS Approach for Dystrophin and Microdystrophin Quantification in Tissue Samples

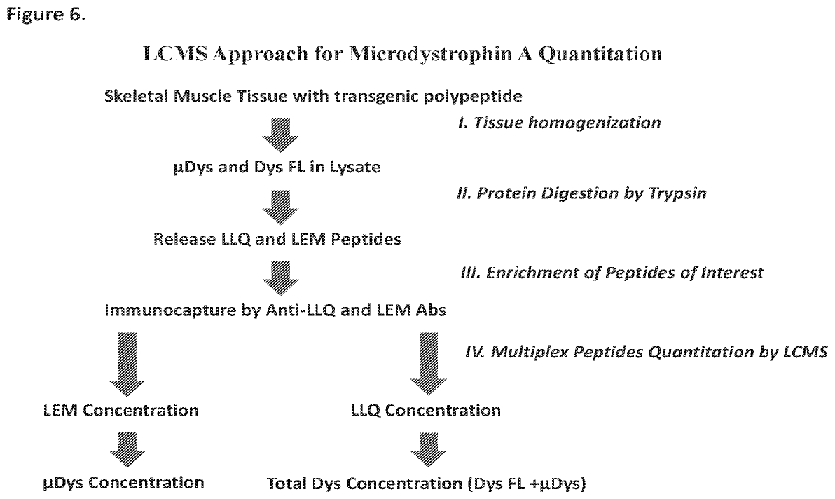

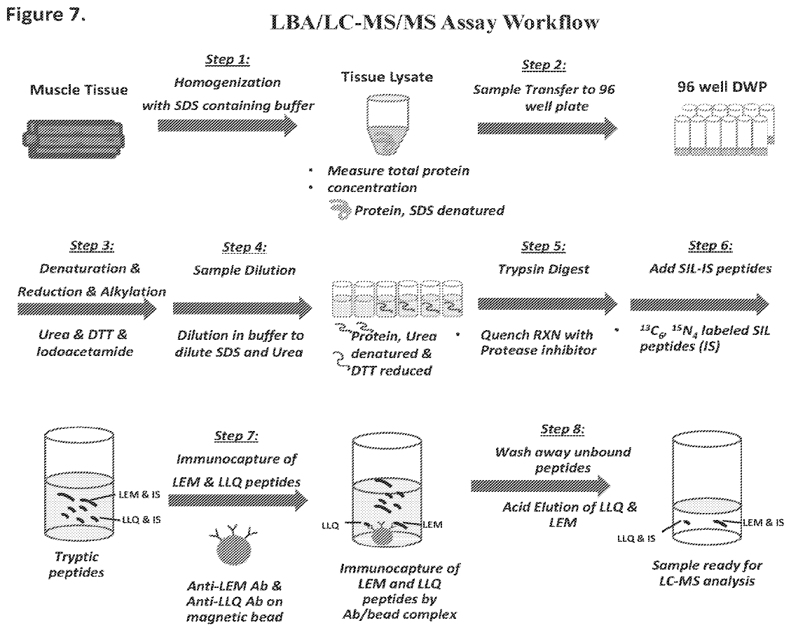

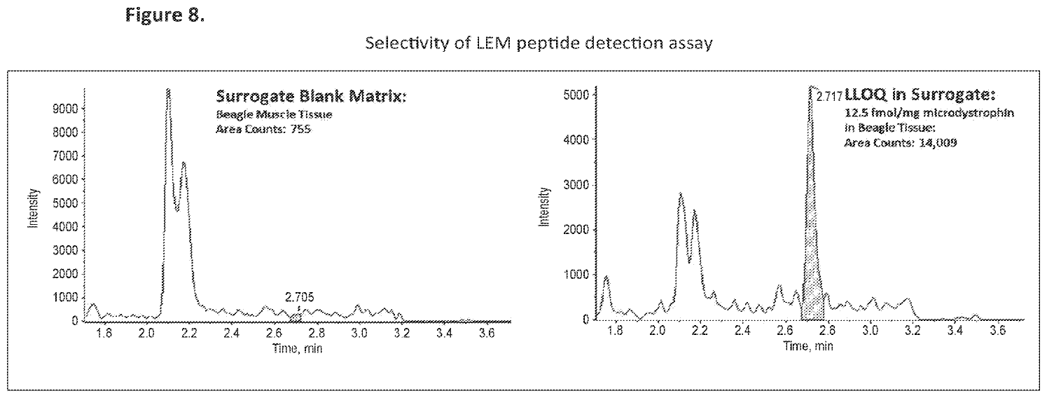

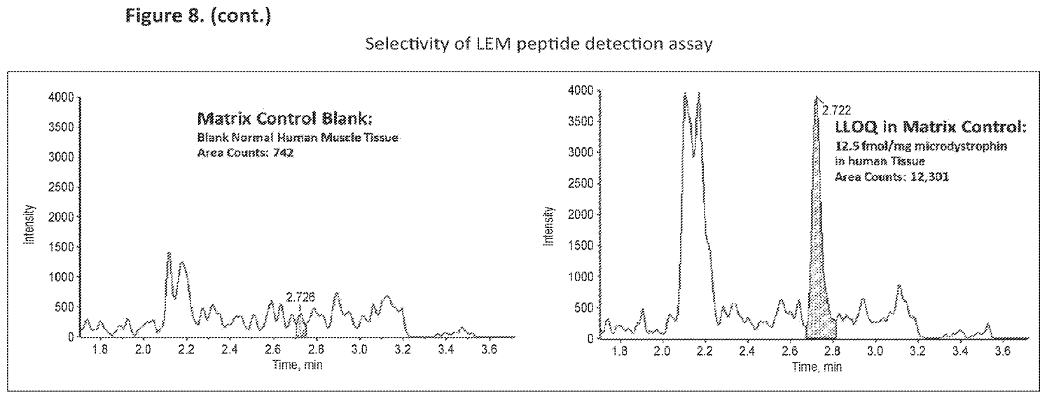

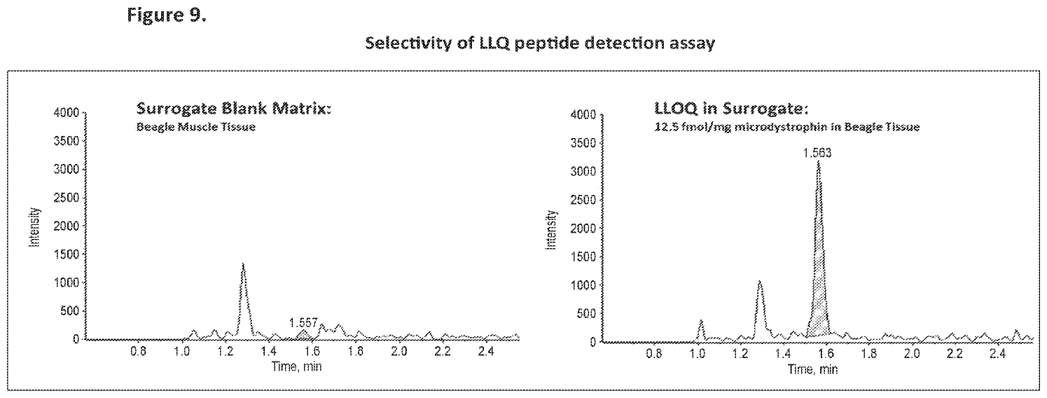

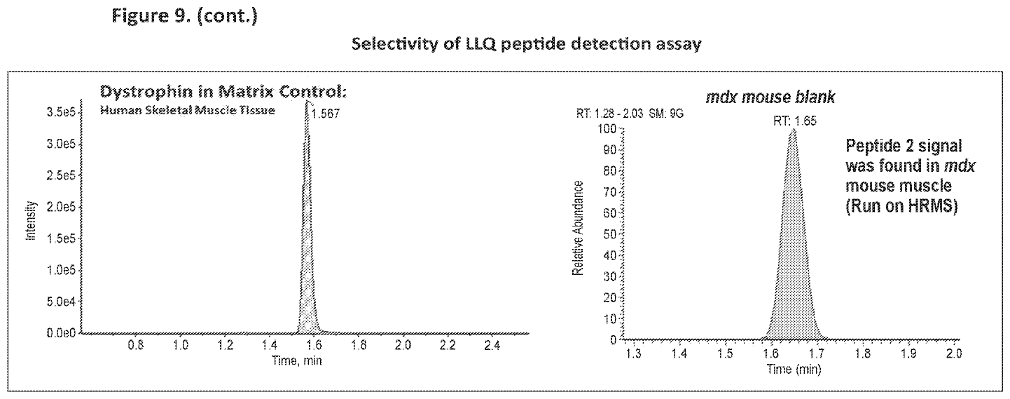

Anti-LEM and anti-LLQ antibodies were used in an LCMS assay to quantify dystrophin and microdystrophin expression levels in various tissue samples. The general outline and workflow of the assay are shown in FIGS. 6 and 7, respectively. The assay allows for simultaneous quantification of both microdystrophin A and dystrophin. The assays were performed using the 130D2-1 anti-LEM and 133D7-1 anti-LLQ monoclonal antibodies. A recombinant microdystrophin (SEQ ID NO: 27) was used as standard to develop a quantification method for AAV transgene encoded microdystrophin. Beagle skeletal muscle was used as surrogate matrix, and normal human skeletal muscle with spiked microdystrophin standard was used as matrix control. Biotinylated monoclonal anti-LEM and anti-LLQ antibodies immobilized on streptavidin magnetic beads were used as immunocapture reagents. Reverse-Phased UHPLC chromatographic separation and a Sciex 7500 Triplequad MS or Thermo Q-Exactive HF-X MS were used to perform the assays. The assay employed stable isotope labeled internal standard (SIL-IS) peptides to facilitate target quantification. LEM peptide detection was used for absolute quantification of microdystrophin; LEM peptide is unique to microdystrophin. LLQ peptide detection was used for quantification of total dystrophin (microdystrophin and dystrophin). LLQ peptide is present in micro- and full-length dystrophin; it is also conserved in mouse, human and NHP dystrophin. Relative quantification of total dystrophin was against full-length dystrophin present in skeletal muscles from normal control subjects. Calibration range for both the LEM and LLQ peptides were 12.5 to 2500 fmol/mg of lysate protein. Precision and Accuracy of QC samples spiked with recombinant microdystrophin in surrogate matrix and normal human matrix were within 20%. Reproducible recovery of both peptides were observed at low and high microdystrophin spiking levels indicating efficient trypsin digest and immunocapture of the peptides. LEM peptide recovery was 262% at low and 126% at high spiking levels (CV<9.2%, n=3; observed recovery yields higher than 100% were due to the non-specific binding loss of LEM peptides in post-spiking solutions), and LLQ peptide recovery was 92% at low and 75% at high spiking levels (CV<7.6%, n=3). LEM and LLQ peptide detection assays were highly selective for microdystrophin and total dystrophin as shown in FIGS. 8 and 9. The LBA/LC-MS assay has the capability to quantify both the transgene encoded microdystrophin and full-length dystrophin protein in various species and different muscle types. The assay has sensitivity, precision and accuracy that are suitable for supporting gene therapy for muscular dystrophy, e.g., Duchenne muscular dystrophy (DMD).

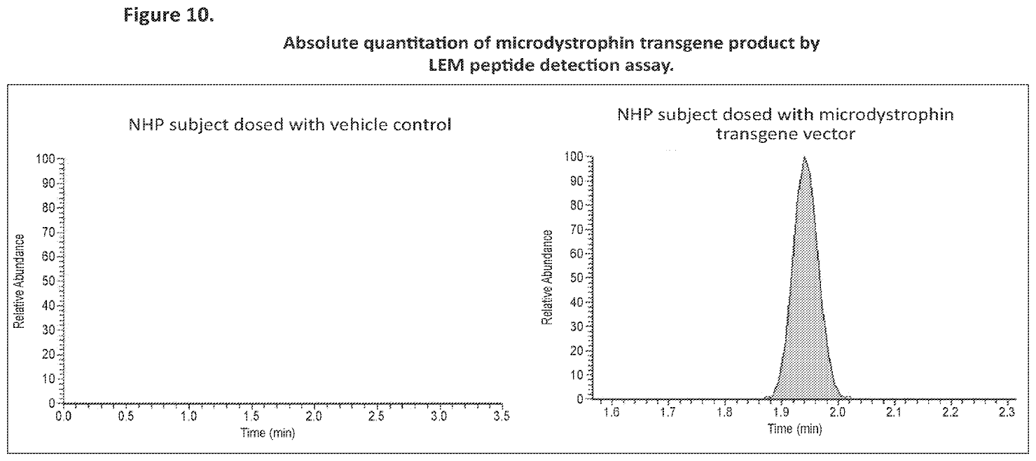

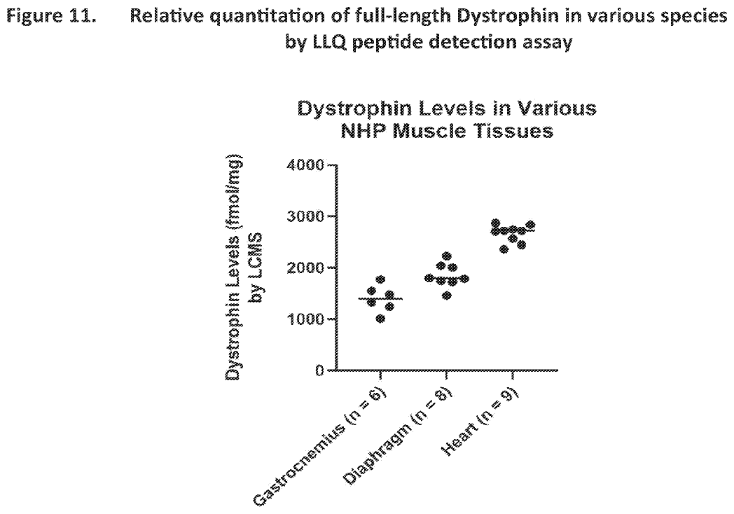

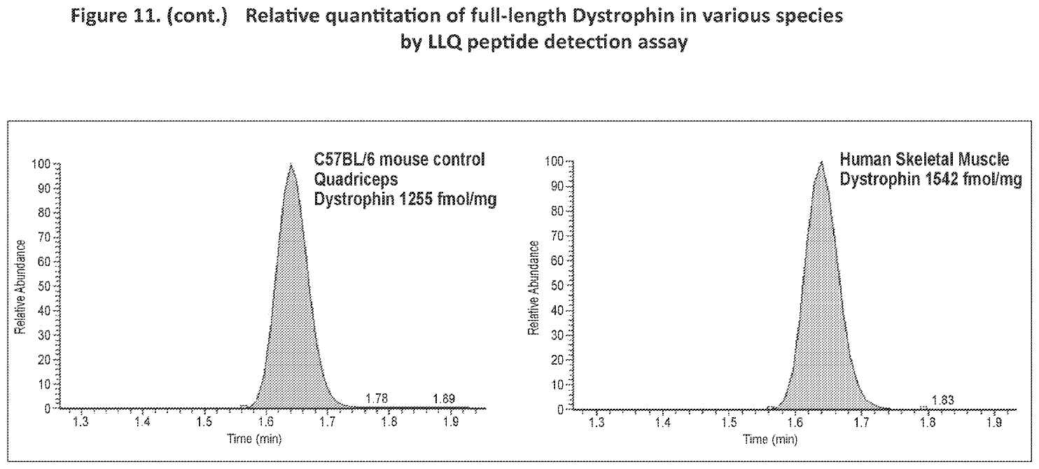

The LBA/LC-MS LEM detection assay was used for highly accurate absolute quantification of microdystrophin levels in muscle samples of non-human primate subjects dosed with recombinant AAV8 particles comprising a microdystrophin transgene (FIG. 10). The LBA/LC-MS LLQ detection assay was used for highly accurate relative quantification of total dystrophin levels in muscle samples from various species (FIG. 11).

While the disclosed methods have been described in connection with what is presently considered to be the most practical and preferred embodiments, it is to be understood that the methods encompassed by the disclosure are not to be limited to the disclosed embodiments, but on the contrary, is intended to cover various modifications and equivalent arrangements included within the spirit and scope of the appended claims.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

SEQUENCES

SEQ ID NO: 1-LEM peptide
LEMPSSLMLEVPTLER
SEQ ID NO: 2
LEMPSSLMLEVP
SEQ ID NO: 3-LLQ peptide
LLQVAVEDR
SEQ ID NO: 4-130D2-1 VH
QSLAESGGRLVTPGTPLTLTCTVSGVDLSYYPMTWVRQAPGKGLEYIGIILHNGTSCYARWARGR
FTISKTSTTVELRITSPTTEDTATYFCARASVASIVGSSDIWGPGTLVTVSS
SEQ ID NO: 5-130D2-1 VL
LVLTQTPSSVSAAVGGTVTINCQSSQSVYKNSALSWYQQKPGQPPKLLIYGASTLASGVPSRFSG
NGSGTQFTLTISGVQCADAATYYCTGAINDEIHAFGGGTEVVVR
SEQ ID NO: 6-130D2-1 VH
CAGTCGCTGGCGGAGTCCGGGGGTCGCCTGGTCACGCCTGGGACACCCCTGACACTCACCTGCAC
AGTCTCTGGAGTCGACCTCAGTTACTATCCAATGACCTGGGTCCGCCAGGCTCCAGGGAAGGGGC
TGGAATACATCGGAATCATTCTTCACAATGGCACCAGTTGCTACGCGCGCTGGGCGAGAGGCCGA
TTCACCATCTCCAAAACCTCGACCACGGTGGAGCTGAGAATCACCAGTCCGACAACCGAGGACAC
GGCCACCTATTTCTGTGCCAGAGCATCTGTTGCTAGTATTGTTGGTTCCAGTGATATCTGGGGCC
CAGGCACCCTGGTCACCGTCTCCTCA
SEQ ID NO: 7-130D2-1 VL
CTAGTGCTGACCCAGACTCCATCCTCCGTGTCTGCGGCTGTGGGAGGCACAGTCACCATCAATTG
CCAGTCCAGTCAGAGTGTTTATAAGAACAGCGCCTTATCCTGGTATCAGCAGAAACCAGGGCAGC
CTCCCAAGCTCCTGATCTATGGTGCATCCACTCTGGCATCTGGGGTCCCATCACGGTTCAGCGGC
AATGGATCTGGGACACAGTTCACTCTCACCATCAGTGGCGTGCAGTGTGCCGATGCTGCCACTTA
CTACTGTACAGGCGCTATTAATGATGAGATACATGCTTTCGGCGGAGGGACCGAGGTGGTGGTCA
GA
SEQ ID NO: 8-130D2-1 VH CDR1
YYPMT
SEQ ID NO: 9-130D2-1 VH CDR2
IILHNGTSCYARWARG
SEQ ID NO: 10-130D2-1 VH CDR3
ASVASIVGSSDI
SEQ ID NO: 11-130D2-1 VL CDR1
QSSQSVYKNSALS
SEQ ID NO: 12-130D2-1 VL CDR2
GASTLAS
SEQ ID NO: 13-130D2-1 VL CDR3
TGAINDEIHA
SEQ ID NO: 14-133D7-1 VH
QSVKESEGGLFKPTDTLTLTCTASGFTISNNAIDWVRQAPGNGLEYIGTIGKSGSAYYASWAKSR
STITRNTNLNTVTLKMTSLTPADTATYFCARLPISKPDTLNLWGPGTLVTVSS
SEQ ID NO: 15-133D7-1 VL
AVLTQTPSPVSAAVGGTVTINCQSSQSVYKNYLSWFQQKPGQPPKLLIYGASTLASGVPSRFKGS
GSGTQFTLTISDVQCDDAATYYCLGGYDTSIDIFTFGGGTEVVVK
SEQ ID NO: 16-133D7-1 VH
CAGTCAGTGAAGGAGTCCGAGGGAGGTCTCTTCAAGCCAACGGATACCCTGACACTCACCTGTAC
AGCCTCCGGATTCACCATCAGTAACAATGCAATAGACTGGGTCCGCCAGGCTCCAGGGAACGGGC
TGGAATATATCGGAACCATTGGTAAAAGTGGTAGCGCATACTACGCGAGCTGGGCGAAAAGCCGA
TCCACCATCACCAGAAACACCAACCTAAACACGGTGACTCTGAAAATGACCAGTCTGACGCCCGC
GGACACGGCCACCTATTTCTGTGCGAGACTTCCTATTTCGAAACCTGATACCCTTAATCTGTGGG
GCCCAGGCACCCTGGTCACC GTCTCCTCA
SEQ ID NO: 17-133D7-1 VL
GCCGTGCTGACCCAGACTCCATCTCCCGTGTCTGCAGCTGTGGGAGGCACAGTCACCATCAATTG
CCAGTCCAGTCAGAGTGTTTATAAGAACTACTTATCCTGGTTTCAGCAGAAACCAGGGCAGCCTC
CCAAGCTCCTGATCTATGGTGCGTCCACTCTGGCATCTGGGGTCCCATCGCGGTTCAAAGGCAGT
GGATCTGGGACACAGTTCACTCTCACCATCAGCGACGTGCAGTGTGACGATGCTGCCACTTACTA
CTGTCTAGGCGGTTATGATACTAGTATTGATATATTTACTTTCGGCGGAGGGACCGAGGTGGTGG
TCAAA
SEQ ID NO: 18-133D7-1 VH CDR1
NNAID
SEQ ID NO: 19-133D7-1 VH CDR2
TIGKSGSAYYASWAKS
SEQ ID NO: 20-133D7-1 VH CDR3
LPISKPDTLNL
SEQ ID NO: 21-133D7-1 VL CDR1
QSSQSVYKNYLS
SEQ ID NO: 22-133D7-1 VL CDR2
GASTLAS
SEQ ID NO: 23-133D7-1 VL CDR3
LGGYDTSIDIFT
SEQ ID NO: 24
PVVTKETAISKLEMPSSLMLEVPALADFNRAWTELTDWLSL
SEQ ID NO: 25
LEMPSSLMLEVPALADFNR
SEQ ID NO: 26
PVVTKETAISKLEMPSSLMLEVPTLERLQELQ
SEQ ID NO: 27
MLWWEEVEDCYEREDVQKKTFTKWVNAQFSKFGKQHIENLFSDLQDGRRLLDLLEGLTGQKLPKE
KGSTRVHALNNVNKALRVLQNNNVDLVNIGSTDIVDGNHKLTLGLIWNIILHWQVKNVMKNIMAG
LQQTNSEKILLSWVRQSTRNYPQVNVINFTTSWSDGLALNALIHSHRPDLFDWNSVVCQQSATQR
LEHAFNIARYQLGIEKLLDPEDVDTTYPDKKSILMYITSLFQVLPQQVSIEAIQEVEMLPRPPKV
TKEEHFQLHHQMHYSQQITVSLAQGYERTSSPKPRFKSYAYTQAAYVTTSDPTRSPFPSQHLEAP
EDKSFGSSLMESEVNLDRYQTALEEVLSWLLSAEDTLQAQGEISNDVEVVKDQFHTHEGYMMDLT
AHQGRVGNILQLGSKLIGTGKLSEDEETEVQEQMNLLNSRWECLRVASMEKQSNLHRVLMDLQNQ
KLKELNDWLTKTEERTRKMEEEPLGPDLEDLKRQVQQHKVLQEDLEQEQVRVNSLTHMVVVVDES
SGDHATAALEEQLKVLGDRWANICRWTEDRWVLLQDILLKWQRLTEEQCLFSAWLSEKEDAVNKI
HTTGFKDQNEMLSSLQKLAVLKADLEKKKQSMGKLYSLKQDLLSTLKNKSVTQKTEAWLDNFARC
WDNLVQKLEKSTAQISQQPDLAPGLTTIGASPTQTVTLVTQPVVTKETAISKLEMPSSLMLEVPT
LERLQELQEATDELDLKLRQAEVIKGSWQPVGDLLIDSLQDHLEKVKALRGEIAPLKENVSHVND
LARQLTTLGIQLSPYNLSTLEDLNTRWKLLQVAVEDRVRQLHEAHRDFGPASQHFLSTSVQGPWE
RAISPNKVPYYINHETQTTCWDHPKMTELYQSLADLNNVRFSAYRTAMKLRRLQKALCLDLLSLS
AACDALDQHNLKQNDQPMDILQIINCLTTIYDRLEQEHNNLVNVPLCVDMCLNWLLNVYDTGRTG
RIRVLSFKTGIISLCKAHLEDKYRYLFKQVASSTGFCDQRRLGLLLHDSIQIPRQLGEVASFGGS
NIEPSVRSCFQFANNKPEIEAALFLDWMRLEPQSMVWLPVLHRVAAAETAKHQAKCNICKECPII
GFRYRSLKHFNYDICQSCFFSGRVAKGHKMHYPMVEYCTPTTSGEDVRDFAKVLKNKFRTKRYFA
KHPRMGYLPVQTVLEGDNMETPVTLINFWPVDSAPASSPQLSHDDTHSRIEHYASRLAEMENSNG
SYLNDSISPNESIDDEHLLIQHYCQSLNQDSPLSQPRSPAQILISLESEERGELERILADLEEEN
RNLQAEYDRLKQQHEHKGLSPLPSPPEMMPTSPQSPR
SEQ ID NO: 28
XLEMPSSLMLEVPTLER, wherein X is R or L
SEQ ID NO: 29
XLLQVAVEDR, wherein X is R or L

What is claimed is:

1. An isolated antibody or antigen binding fragment thereof capable of binding to a polypeptide comprising the amino acid sequence of SEQ ID NO: 1.

2. The antibody or antigen binding fragment thereof of claim 1, wherein the amino acid sequence of the polypeptide consist of SEQ ID NO: 1.

3. The antibody or antigen binding fragment thereof of claim 1 or claim 2 that does not bind to a peptide consisting of the amino acid of SEQ ID NO: 2.

4. The antibody or antigen binding fragment thereof of any one of claims 1 to 3, wherein the antibody is a polyclonal antibody.

5. The antibody or antigen binding fragment thereof of any one of claims 1 to 3, wherein the antibody is a monoclonal antibody.

6. An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
a) the VH CDR1, CDR2 and CDR3 and the VL CDR1, CDR2 and CDR3 of the 130D2-1 antibody, respectively, independently comprising 0, 1, 2, 3, 4 or 5 substitutions;
b) the VH CDR1, CDR2 and CDR3 and the VL CDR1, CDR2 and CDR3 of the 133E10-1 antibody, respectively, independently comprising 0, 1, 2, 3, 4 or 5 substitutions; or
c) the VH CDR1, CDR2 and CDR3 and the VL CDR1, CDR2 and CDR3 of the 75A2-1 antibody, respectively, independently comprising 0, 1, 2, 3, 4 or 5 substitutions.

7. An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
a) the VH CDR1, CDR2 and CDR3 and the VL CDR1, CDR2 and CDR3 of the 130D2-1 antibody, respectively;
b) the VH CDR1, CDR2 and CDR3 and the VL CDR1, CDR2 and CDR3 of the 133E10-1 antibody, respectively; or
c) the VH CDR1, CDR2 and CDR3 and the VL CDR1, CDR2 and CDR3 of the 75A2-1 antibody, respectively.

8. The isolated antibody or antigen binding fragment thereof according to claim 6 or claim 7, wherein the VH CDR1, CDR2 and CDR3 and the VL CDR1, CDR2 and CDR3 of 130D2-1, 133E10-1 and 75A2-1 are according to Kabat.

9. An isolated antibody or antigen binding fragment thereof comprising a variable heavy chain domain (VH) and a variable light chain domain (VL), wherein the VH and VL comprises
a) an amino acid sequence having at least 70%, at least 80%, at least 90%, at least 95%, at least 97% or 100% identity with the VH and VL of the 130D2-1 antibody, respectively;
b) an amino acid sequence having at least 70%, at least 80%, at least 90%, at least 95%, at least 97% or 100% identity with the VH and VL of the 133E10-1 antibody, respectively; or
c) an amino acid sequence having at least 70%, at least 80%, at least 90%, at least 95%, at least 97% or 100% identity with the VH and VL of the 75A2-1 antibody, respectively.

10. An isolated antibody or antigen binding fragment thereof comprising a variable heavy chain domain (VH) and a variable light chain domain (VL), wherein the VH and VL comprises
a) the VH and VL of the 130D2-1 antibody, respectively;
b) the VH and VL of the 133E10-1 antibody, respectively; or
c) the VH and VL of the 75A2-1 antibody, respectively.

11. An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the
a) the VH CDR1 comprises an amino acid sequence of SEQ ID NO: 8 comprising 0, 1, 2, 3, 4 or 5 substitutions;
b) the VH CDR2 comprises an amino acid sequence of SEQ ID NO: 9 comprising 0, 1, 2, 3, 4 or 5 substitutions;
c) the VH CDR3 comprises an amino acid sequence of SEQ ID NO: 10 comprising 0, 1, 2, 3, 4 or 5 substitutions;
d) the VL CDR1 comprises an amino acid sequence of SEQ ID NO: 11 comprising 0, 1, 2, 3, 4 or 5 substitutions;
e) the VL CDR2 comprises an amino acid sequence of SEQ ID NO: 12 comprising 0, 1, 2, 3, 4 or 5 substitutions; and
f) the VL CDR3 comprises an amino acid sequence of SEQ ID NO: 13 comprising 0, 1, 2, 3, 4 or 5 substitutions.

12. An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the
a) the VH CDR1 comprises the amino acid sequence of SEQ ID NO: 8;
b) the VH CDR2 comprises the amino acid sequence of SEQ ID NO: 9;
c) the VH CDR3 comprises the amino acid sequence of SEQ ID NO: 10;
d) the VL CDR1 comprises the amino acid sequence of SEQ ID NO: 11;
e) the VL CDR2 comprises the amino acid sequence of SEQ ID NO: 12; and
f) the VL CDR3 comprises the amino acid sequence of SEQ ID NO: 13.

13. An isolated antibody or antigen binding fragment thereof comprising a variable heavy chain domain (VH) and a variable light chain domain (VL), wherein
a) the VH comprises an amino acid sequence having at least 70%, at least 80%, at least 90%, at least 95%, at least 97% or 100% identity with SEQ ID NO: 4; and
b) the VL comprises an amino acid sequence having at least 70%, at least 80%, at least 90%, at least 95%, at least 97% or 100% identity with SEQ ID NO: 5.

14. An isolated antibody or antigen binding fragment thereof comprising a variable heavy chain domain (VH) and a variable light chain domain (VL), wherein
a) the VH comprises the amino acid sequence of SEQ ID NO: 4; and
b) the VL comprises the amino acid sequence of SEQ ID NO: 5.

15. The antibody or antigen binding fragment thereof of any one of claims 6 to 14 capable of binding to a polypeptide comprising the amino acid sequence of SEQ ID NO: 1.

16. The antibody or antigen binding fragment thereof of claim 15, wherein the amino acid sequence of the polypeptide consist of SEQ ID NO: 1.

17. The antibody or antigen binding fragment thereof of any one of claims 6 to 16 that does not bind to a peptide consisting of the amino acid of SEQ ID NO: 2.

18. An isolated antibody or antigen binding fragment thereof capable of binding to a polypeptide comprising the amino acid sequence of SEQ ID NO: 3.

19. The antibody or antigen binding fragment thereof of claim 18, wherein the amino acid sequence of the polypeptide consist of SEQ ID NO: 3.

20. The antibody or antigen binding fragment thereof of claim 18 or claim 19, wherein the antibody is a polyclonal antibody.

21. The antibody or antigen binding fragment thereof of claim 18 or claim 19, wherein the antibody is a monoclonal antibody.

22. An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
a) the VH CDR1, CDR2 and CDR3 and the VL CDR1, CDR2 and CDR3 of the 112E4-1 antibody, respectively, independently comprising 0, 1, 2, 3, 4 or 5 substitutions;
b) the VH CDR1, CDR2 and CDR3 and the VL CDR1, CDR2 and CDR3 of the 115G6-1 antibody, respectively, independently comprising 0, 1, 2, 3, 4 or 5 substitutions;
c) the VH CDR1, CDR2 and CDR3 and the VL CDR1, CDR2 and CDR3 of the 119H2-1 antibody, respectively, independently comprising 0, 1, 2, 3, 4 or 5 substitutions;
d) the VH CDR1, CDR2 and CDR3 and the VL CDR1, CDR2 and CDR3 of the 121F10-1 antibody, respectively, independently comprising 0, 1, 2, 3, 4 or 5 substitutions; or
e) the VH CDR1, CDR2 and CDR3 and the VL CDR1, CDR2 and CDR3 of the 133D7-1 antibody, respectively, independently comprising 0, 1, 2, 3, 4 or 5 substitutions.

23. An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the VH CDR1, CDR2 and CDR3 and the VL CDR1, CDR2 and CDR3 comprises
a) the VH CDR1, CDR2 and CDR3 and the VL CDR1, CDR2 and CDR3 of the 112E4-1 antibody, respectively;
b) the VH CDR1, CDR2 and CDR3 and the VL CDR1, CDR2 and CDR3 of the 115G6-1 antibody, respectively;
c) the VH CDR1, CDR2 and CDR3 and the VL CDR1, CDR2 and CDR3 of the 119H2-1 antibody, respectively;
d) the VH CDR1, CDR2 and CDR3 and the VL CDR1, CDR2 and CDR3 of the 121F10-1 antibody, respectively; or
e) the VH CDR1, CDR2 and CDR3 and the VL CDR1, CDR2 and CDR3 of the 133D7-1 antibody, respectively.

24. The isolated antibody or antigen binding fragment thereof according to claim 22 or claim 23, wherein the VH CDR1, CDR2 and CDR3 and the VL CDR1, CDR2 and CDR3 of 112E4-1, 115G6-1, 119H2-1, 121F10-1 and 133D7-1 are according to Kabat.

25. An isolated antibody or antigen binding fragment thereof comprising a variable heavy chain domain (VH) and a variable light chain domain (VL), wherein the VH and VL comprises
a) an amino acid sequence having at least 70%, at least 80%, at least 90%, at least 95%, at least 97% or 100% identity with the VH and VL of the 112E4-1 antibody, respectively;
b) an amino acid sequence having at least 70%, at least 80%, at least 90%, at least 95%, at least 97% or 100% identity with the VH and VL of the 115G6-1 antibody, respectively;
c) an amino acid sequence having at least 70%, at least 80%, at least 90%, at least 95%, at least 97% or 100% identity with the VH and VL of the 119H2-1 antibody, respectively;
d) an amino acid sequence having at least 70%, at least 80%, at least 90%, at least 95%, at least 97% or 100% identity with the VH and VL of the 121F10-1 antibody, respectively; or
e) an amino acid sequence having at least 70%, at least 80%, at least 90%, at least 95%, at least 97% or 100% identity with the VH and VL of the 133D7-1 antibody, respectively.

26. An isolated antibody or antigen binding fragment thereof comprising a variable heavy chain domain (VH) and a variable light chain domain (VL), wherein the VH and VL comprises
a) the VH and VL of the 112E4-1 antibody, respectively;
b) the VH and VL of the 115G6-1 antibody, respectively;
c) the VH and VL of the 119H2-1 antibody, respectively;
d) the VH and VL of the 121F10-1 antibody, respectively; or
e) the VH and VL of the 133D7-1 antibody, respectively.

27. An isolated antibody or antigen binding fragment thereof comprising a variable heavy chain domain (VH) and a variable light chain domain (VL), wherein the VH comprises VH complementarity determining region 1 (CDR1), CDR2 and CDR3 and the VL comprises VL CDR1, CDR2 and CDR3, wherein the
a) the VH CDR1 comprises an amino acid sequence of SEQ ID NO: 18 comprising 0, 1, 2, 3, 4 or 5 substitutions;
b) the VH CDR2 comprises an amino acid sequence of SEQ ID NO: 19 comprising 0, 1, 2, 3, 4 or 5 substitutions;
c) the VH CDR3 comprises an amino acid sequence of SEQ ID NO: 20 comprising 0, 1, 2, 3, 4 or 5 substitutions;
d) the VL CDR1 comprises an amino acid sequence of SEQ ID NO: 21 comprising 0, 1, 2, 3, 4 or 5 substitutions;
e) the VL CDR2 comprises an amino acid sequence of SEQ ID NO: 22 comprising 0, 1, 2, 3, 4 or 5 substitutions; and
f) the VL CDR3 comprises an amino acid sequence of SEQ ID NO: 23 comprising 0, 1, 2, 3, 4 or 5 substitutions.

28. An isolated antibody or antigen binding fragment thereof comprising a variable heavy chain domain (VH) and a variable light chain domain (VL), wherein the VH comprises VH complementarity determining regions (CDRs) 1, 2 and 3 and the VL comprises VL CDRs 1, 2 and 3, wherein the
a) the VH CDR1 comprises the amino acid sequence of SEQ ID NO: 18;
b) the VH CDR2 comprises the amino acid sequence of SEQ ID NO: 19;
c) the VH CDR3 comprises the amino acid sequence of SEQ ID NO: 20;
d) the VL CDR1 comprises the amino acid sequence of SEQ ID NO: 21;
e) the VL CDR2 comprises the amino acid sequence of SEQ ID NO: 22; and
f) the VL CDR3 comprises the amino acid sequence of SEQ ID NO: 23.

29. An isolated antibody or antigen binding fragment thereof comprising a variable heavy chain domain (VH) and a variable light chain domain (VL), wherein
a) the VH comprises an amino acid sequence having at least 70%, at least 80%, at least 90%, at least 95%, at least 97% or 100% identity with SEQ ID NO: 14; and
b) the VL comprises an amino acid sequence having at least 70%, at least 80%, at least 90%, at least 95%, at least 97% or 100% identity with SEQ ID NO: 15.

30. An isolated antibody or antigen binding fragment thereof comprising a variable heavy chain domain (VH) and a variable light chain domain (VL), wherein
a) the VH comprises the amino acid sequence of SEQ ID NO: 14; and
b) the VL comprises the amino acid sequence of SEQ ID NO: 15.

31. The antibody or antigen binding fragment thereof of any one of claims 22 to 30 capable of binding to a polypeptide comprising the amino acid sequence of SEQ ID NO: 3.

32. The antibody or antigen binding fragment thereof of claim 31, wherein the amino acid sequence of the polypeptide consist of SEQ ID NO: 3.

33. The antibody or antigen binding fragment thereof of any one of claims 1 to 32, wherein the antibody fragment comprises a single-chain Fv (scFv), F (ab) fragment, F(ab′) 2 fragment, or an isolated VH domain.

34. A composition comprising the antibody or antigen binding fragment thereof of any one of claims 1 to 33.

35. The composition of claim 34 that is a pharmaceutical composition further comprising a pharmaceutically acceptable excipient.

36. An affinity resin comprising the antibody or antigen binding fragment thereof of any one of claims 1 to 33 and a solid support.

37. The affinity resin of claim 36, wherein the solid support comprises a bead, gelatin, or agarose.

38. The affinity resin of claim 36 or claim 37, wherein the antibody or antigen binding fragment thereof is attached to the solid support by covalent bonding.

39. The affinity resin of claim 36 or claim 37, wherein the antibody or antigen binding fragment thereof is attached to the solid support by non-covalent association.

40. An isolated polynucleotide encoding the antibody or antigen binding fragment thereof of any one of claims 1 to 33.

41. A vector comprising the polynucleotide of claim 40.

42. A host cell comprising the polynucleotide of claim 40 or the vector of claim 41.

43. The host cell of claim 42 which is a CHO cell or a HEK293 cell.

44. A method of producing the antibody or antigen binding fragment thereof of any one for claims 1 to 33 comprising incubating the host cell of claim 42 or claim 43 under suitable conditions to produce the antibody or antigen binding fragment thereof.

45. A method of isolating a peptide from a sample comprising
a) contacting the sample comprising the peptide with a composition comprising the antibody or antigen binding fragment thereof of any one for claims 1 to 17, and optionally with a composition comprising the antibody or antigen binding fragment thereof of any one for claims 18 to 33, under conditions that permit binding of the peptide to the antibody or antigen binding fragment thereof;
b) removing a portion of the sample that is not bound to the antibody or antigen binding fragment thereof; and
c) dissociating the peptide from the antibody or antigen binding fragment thereof, wherein the amino acid sequence of the peptide comprises SEQ ID NO: 1 or 3.

46. The method of claim 45, wherein the amino acid sequence of the peptide consist of SEQ ID NO: 1 or 3.

47. The method of claim 45 or claim 46, wherein the composition comprising the antibody or antigen binding fragment thereof is an affinity resin comprising a solid support selected from the group consisting of a bead, gelatin, or agarose.

48. The method of claim 47, wherein the antibody or antigen binding fragment thereof is attached to the solid support by covalent bonding.

49. The method of claim 47, wherein the antibody or antigen binding fragment thereof is attached to the solid support by non-covalent association.

50. The method of any one of claims 45 to 49, wherein the sample comprises a protease digested protein isolate obtained from a subject.

51. The method of claim 50, wherein the sample comprises a protease digested protein isolate obtained from a skeletal muscle tissue of the subject.

52. The method of claim 50 or claim 51, wherein the protease comprises trypsin.

53. The method of any one of claims 50 to 52, wherein the subject is a human, primate, canine or murine subject.

54. The method of any one of claims 50 to 53, wherein the subject has been administered a recombinant polypeptide comprising the amino acid sequence of SEQ ID NO: 28 and/or 29.

55. The method of any one of claims 50 to 53, wherein the subject has been administered a recombinant polynucleotide encoding a recombinant polypeptide comprising the amino acid sequence of SEQ ID NO: 28 and/or 29.

56. The method of any one of claims 50 to 53, wherein the subject has been administered a recombinant virus comprising a polynucleotide encoding a recombinant polypeptide comprising the amino acid sequence of SEQ ID NO: 28 and/or 29.

57. The method of claim 56, wherein the recombinant virus is a recombinant adeno-associated virus.

58. The method of any one of claims 45 to 57, further comprising
d) recovering the peptide; and
e) determining the amount of peptide recovered in step d).

59. The method of claim 58, wherein the amount of peptide is determined by LC/MS or LC-MS/MS.

60. The method of any one of claims 45 to 59, wherein the sample further comprises a stable isotope labeled peptide standard comprising the amino acid sequence of SEQ ID NO: 1 or 3.

61. The method of any one of claims 54 to 60, wherein the recombinant polypeptide is microdystrophin.

62. The method of claim 61, wherein the microdystrophin comprises the amino acid sequence of SEQ ID NO: 27.

63. A method of quantifying the level of a recombinant polypeptide in a subject comprising:
a) providing a sample comprising a protease digested protein isolate obtained from the subject, wherein the sample comprises one or more peptides having the amino acid sequence of SEQ ID NO: 1 or 3;
b) contacting the sample with a composition comprising the antibody or antigen binding fragment thereof of any one for claims 1 to 17, and optionally with a composition comprising the antibody or antigen binding fragment thereof of any one for claims 18 to 33, under conditions that permit binding of the antibody or antigen binding fragment thereof to the peptide;
c) recovering the peptide bound to the antibody or antigen binding fragment thereof; and
d) determining the amount of peptide recovered in step d),
wherein the amino acid sequence of the recombinant polypeptide comprises SEQ ID NO: 28 and/or 29.

64. The method of claim 63, wherein the amount of peptide is determined by LC/MS or LC-MS/MS.

65. The method of claim 63 or claim 64, wherein the sample comprises a protease digested protein isolate obtained from a skeletal muscle tissue of the subject.

66. The method of any one of claims 63 to 65, wherein the subject is a human, primate, canine or murine subject.

67. The method of any one of claims 63 to 66, wherein the subject has been administered the recombinant polypeptide comprising the amino acid sequence of SEQ ID NO: 28 and/or 29.

68. The method of any one of claims 63 to 66, wherein the subject has been administered a recombinant polynucleotide encoding a recombinant polypeptide comprising the amino acid sequence of SEQ ID NO: 28 and/or 29.

69. The method of any one of claims 63 to 66, wherein the subject has been administered a recombinant virus comprising a polynucleotide encoding a recombinant polypeptide comprising the amino acid sequence of SEQ ID NO: 28 and/or 29.

70. The method of claim 69, wherein the recombinant virus is a recombinant adeno-associated virus.

71. The method of any one of claims 63 to 70, wherein the recombinant polypeptide is microdystrophin.

72. The method of claim 71, wherein the microdystrophin comprises the amino acid sequence of SEQ ID NO: 27.

73. The method of any one of claims 63 to 72, wherein the sample further comprises a labeled peptide or peptides that are capable of binding to the antibody or antigen binding fragment thereof.

74. The method of claim 73, wherein the labeled peptide or peptides are a stable isotope labeled peptide or peptides.

75. A method of quantifying the level of dystrophin and/or microdystrophin expression in a subject comprising:
a) providing a sample comprising a protease digested protein isolate obtained from the subject, wherein the sample comprises one or more peptides having the amino acid sequence of SEQ ID NO: 1 or 3;
b) contacting the sample with a composition comprising the antibody or antigen binding fragment thereof of any one for claims 1 to 17, and optionally with a composition comprising the antibody or antigen binding fragment thereof of any one for claims 18 to 33, under conditions that permit binding of the antibody or antigen binding fragment thereof to the peptide;
c) recovering the peptide bound to the antibody or antigen binding fragment thereof; and
d) determining the amount of peptide recovered in step d),
wherein the amino acid sequence of the microdystrophin comprises SEQ ID NO: 28 and/or 29.

76. The method of claim 75, wherein the amount of peptide is determined by LC/MS or LC-MS/MS.

77. The method of claim 75 or claim 76, which provides an absolute quantification of the level of dystrophin and/or microdystrophin expression.

78. The method of claim 75 or claim 76, which provides a relative quantification of the level of dystrophin and/or microdystrophin expression.

79. The method of any one of claims 75 to 78, wherein the sample comprises a protease digested protein isolate obtained from a skeletal muscle tissue of the subject.

80. The method of any one of claims 75 to 79, wherein the protease is trypsin.

81. The method of any one of claims 75 to 80, wherein the sample further comprises a labeled peptide or peptides that are capable of binding to the antibody or antigen binding fragment thereof.

82. The method of claim 81, wherein the labeled peptide or peptides are a stable isotope labeled peptide or peptides.

83. The method of claim 81 or claim 82, wherein the labeled peptide or peptides comprise the amino acid sequence of SEQ ID NO: 1 or 3.

84. The method of any one of claims 75 to 83, wherein the subject is a human, primate, canine or murine subject.

85. The method of claim 84, wherein the subject is a human.

86. The method of claim 84, wherein the subject is a primate.

87. The method of claim 84, wherein the subject is a murine.

88. The method of any one of claims 75 to 84, wherein the subject suffers from Duchenne muscular dystrophy.

89. The method of any one of claims 75 to 84, wherein the subject is a non-human mammal that has been genetically modified to comprise one or more mutations in the dystrophin gene.

90. The method of any one of claims 75 to 89, wherein the subject has been administered a recombinant polynucleotide encoding a microdystrophin comprising the amino acid sequence of SEQ ID NO: 28 and 29.

91. The method of claim 90, wherein the microdystrophin comprises the amino acid sequence of SEQ ID NO: 27.

92. The method of claim 90 or claim 91, wherein the recombinant polynucleotide is DNA.

93. The method of claim 90 or claim 91, wherein the recombinant polynucleotide is RNA.

94. The method of claim 93, wherein the RNA is mRNA comprising a modified ribonucleotide.

95. The method of any one of claims 75 to 89, wherein the subject has been administered a recombinant virus comprising a polynucleotide encoding a microdystrophin comprising the amino acid sequence of SEQ ID NO: 28 and 29.

96. The method of claim 69, wherein the microdystrophin comprises the amino acid sequence of SEQ ID NO: 27.

97. The method of claim 95 or claim 96, wherein the recombinant virus is a recombinant adeno-associated virus.


SEQUENCE LISTING (29 sequences)

SEQ ID NO: 1 - protein, 16 aa, synthetic construct
LEMPSSLMLEVPTLER

SEQ ID NO: 2 - protein, 12 aa, synthetic construct
LEMPSSLMLEVP

SEQ ID NO: 3 - protein, 9 aa, synthetic construct
LLQVAVEDR

SEQ ID NO: 4 - protein, 117 aa, synthetic construct
QSLAESGGRLVTPGTPLTLTCTVSGVDLSYYPMTWVRQAPGKGLEYIGIILHNGTSCYARWARGRFTISKTSTTVELRITSPTTEDTATYFCARASVASIVGSSDIWGPGTLVTVSS

SEQ ID NO: 5 - protein, 109 aa, synthetic construct
LVLTQTPSSVSAAVGGTVTINCQSSQSVYKNSALSWYQQKPGQPPKLLIYGASTLASGVPSRFSGNGSGTQFTLTISGVQCADAATYYCTGAINDEIHAFGGGTEVVVR

SEQ ID NO: 6 - DNA, 351 bp, synthetic construct
  source 1..351: mol_type = other DNA
cagtcgctggcggagtccgggggtcgcctggtcacgcctgggacacccctgacactcacctgcacagtctctggagtcgacctcagttactatccaatgacctgggtccgccaggctccagggaaggggctggaatacatcggaatcattcttcacaatggcaccagttgctacgcgcgctgggcgagaggccgattcaccatctccaaaacctcgaccacggtggagctgagaatcaccagtccgacaaccgaggacacggccacctatttctgtgccagagcatctgttgctagtattgttggttccagtgatatctggggcccaggcaccctggtcaccgtctcctca

SEQ ID NO: 7 - DNA, 327 bp, synthetic construct
  source 1..327: mol_type = other DNA
ctagtgctgacccagactccatcctccgtgtctgcggctgtgggaggcacagtcaccatcaattgccagtccagtcagagtgtttataagaacagcgccttatcctggtatcagcagaaaccagggcagcctcccaagctcctgatctatggtgcatccactctggcatctggggtcccatcacggttcagcggcaatggatctgggacacagttcactctcaccatcagtggcgtgcagtgtgccgatgctgccacttactactgtacaggcgctattaatgatgagatacatgctttcggcggagggaccgaggtggtggtcaga

SEQ ID NO: 8 - protein, 5 aa, synthetic construct
YYPMT

SEQ ID NO: 9 - protein, 16 aa, synthetic construct
IILHNGTSCYARWARG

SEQ ID NO: 10 - protein, 12 aa, synthetic construct
ASVASIVGSSDI

SEQ ID NO: 11 - protein, 13 aa, synthetic construct
QSSQSVYKNSALS

SEQ ID NO: 12 - protein, 7 aa, synthetic construct
GASTLAS

SEQ ID NO: 13 - protein, 10 aa, synthetic construct
TGAINDEIHA

SEQ ID NO: 14 - protein, 118 aa, synthetic construct
QSVKESEGGLFKPTDTLTLTCTASGFTISNNAIDWVRQAPGNGLEYIGTIGKSGSAYYASWAKSRSTITRNTNLNTVTLKMTSLTPADTATYFCARLPISKPDTLNLWGPGTLVTVSS

SEQ ID NO: 15 - protein, 110 aa, synthetic construct
AVLTQTPSPVSAAVGGTVTINCQSSQSVYKNYLSWFQQKPGQPPKLLIYGASTLASGVPSRFKGSGSGTQFTLTISDVQCDDAATYYCLGGYDTSIDIFTFGGGTEVVVK

SEQ ID NO: 16 - DNA, 354 bp, synthetic construct
  source 1..354: mol_type = other DNA
cagtcagtgaaggagtccgagggaggtctcttcaagccaacggataccctgacactcacctgtacagcctccggattcaccatcagtaacaatgcaatagactgggtccgccaggctccagggaacgggctggaatatatcggaaccattggtaaaagtggtagcgcatactacgcgagctgggcgaaaagccgatccaccatcaccagaaacaccaacctaaacacggtgactctgaaaatgaccagtctgacgcccgcggacacggccacctatttctgtgcgagacttcctatttcgaaacctgatacccttaatctgtggggcccaggcaccctggtcaccgtctcctca

SEQ ID NO: 17 - DNA, 330 bp, synthetic construct
  source 1..330: mol_type = other DNA
gccgtgctgacccagactccatctcccgtgtctgcagctgtgggaggcacagtcaccatcaattgccagtccagtcagagtgtttataagaactacttatcctggtttcagcagaaaccagggcagcctcccaagctcctgatctatggtgcgtccactctggcatctggggtcccatcgcggttcaaaggcagtggatctgggacacagttcactctcaccatcagcgacgtgcagtgtgacgatgctgccacttactactgtctaggcggttatgatactagtattgatatatttactttcggcggagggaccgaggtggtggtcaaa

SEQ ID NO: 18 - protein, 5 aa, synthetic construct
NNAID

SEQ ID NO: 19 - protein, 16 aa, synthetic construct
TIGKSGSAYYASWAKS

SEQ ID NO: 20 - protein, 11 aa, Synthetic construct
LPISKPDTLNL

SEQ ID NO: 21 - protein, 12 aa, synthetic construct
QSSQSVYKNYLS

SEQ ID NO: 22 - protein, 7 aa, synthetic construct
GASTLAS

SEQ ID NO: 23 - protein, 12 aa, synthetic construct
LGGYDTSIDIFT

SEQ ID NO: 24 - protein, 41 aa, synthetic construct
PVVTKETAISKLEMPSSLMLEVPALADFNRAWTELTDWLSL

SEQ ID NO: 25 - protein, 19 aa, synthetic construct
LEMPSSLMLEVPALADFNR

SEQ ID NO: 26 - protein, 32 aa, synthetic construct
PVVTKETAISKLEMPSSLMLEVPTLERLQELQ

SEQ ID NO: 27 - protein, 1337 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPR

SEQ ID NO: 28 - protein, 17 aa, synthetic construct
  VARIANT 1: note = R or L
XLEMPSSLMLEVPTLER

SEQ ID NO: 29 - protein, 10 aa, synthetic construct
  VARIANT 1: note = R or L
XLLQVAVEDR